(19) United States
(12) Patent Application Publication
(10) Pub. No.: US 2005/0239845 A1
(43) Pub. Date: Oct. 27, 2005

(54) Combination of proton pump inhibitor, buffering agent, and prokinetic agent

(75) Inventors: Gerald T. Proehl, San Diego, CA (US); Warren Hall, Del Mar, CA (US); Kay Olmstead, San Diego, CA (US); Bonnie Hepburn, Escondido, CA (US)

Correspondence Address: WILSON SONSINI GOODRICH & ROSATI, 650 PAGE MILL ROAD, PALO ALTO, CA, 94304-1050, US

(73) Assignee: Santarus, Inc.

(21) Appl. No.: 11/107,349

(22) Filed: Apr. 15, 2005

Related U.S. Application Data
(60) Provisional application No. 60/562,820, filed on Apr. 16, 2004.

(51) Int. Cl. A61K 31/4439

(52) U.S. Cl. 514/338

(57) ABSTRACT

Pharmaceutical compositions comprising a proton pump inhibitor, one or more buffering agent and a prokinetic agent are described. Methods are described for treating gastric acid related disorders, using pharmaceutical compositions comprising a proton pump inhibitor, a buffering agent, and a prokinetic agent.

### CROSS REFERENCE TO RELATED APPLICATIONS

This application claims priority to U.S. Provisional Application Ser. No. 60/562,820 filed Apr. 16, 2004, which is incorporated herein by reference in its entirety.

### FIELD OF THE INVENTION

The present invention is related to pharmaceutical compositions comprising a proton pump inhibitor, a buffering agent, and a prokinetic agent. Methods for manufacture of the pharmaceutical compositions and use of the pharmaceutical compositions in treating disease are disclosed.

### BACKGROUND OF THE INVENTION

Proton Pump Inhibitors

Proton pump inhibitors (PPIs) are a class of acid-labile pharmaceutical compounds that block gastric acid secretion pathways. Exemplary proton pump inhibitors include, omeprazole (Prilosec®), lansoprazole (Prevacid®), esomeprazole (Nexium®), rabeprazole (Aciphex®), pantoprazole (Protonix®), pariprazole, tenatoprazole, and leminoprazole. The drugs of this class suppress gastrointestinal acid secretion by the specific inhibition of the H⁺/K⁺-ATPase enzyme system (proton pump) at the secretory surface of the gastrointestinal parietal cell. Most proton pump inhibitors are susceptible to acid degradation and, as such, are rapidly destroyed in an acidic pH environment in the stomach. Therefore, proton pump inhibitors are often administered as enteric-coated dosage forms in order to permit release of the drug in the duodenum after having passed through the stomach. If the enteric-coating of these formulated products is disrupted (e.g., during trituration to compound a liquid dosage form, or by chewing an enteri-coated granular capsule or tablet), or if a co-administered buffering agent fails to sufficiently neutralize the gastrointestinal pH, the uncoated drug is exposed to stomach acid and may be degraded.

Omeprazole, a substituted bicyclic aryl-imidazole, 5-methoxy-2-[(4-methoxy-3, 5-dimethyl-2-pyridinyl)methyl]sulfinyl]-1H-benzimidazole, is a proton pump inhibitor that inhibits gastrointestinal acid secretion. U.S. Pat. No. 4,786,505 to Lovgren et al. teaches that a pharmaceutical oral solid dosage form of omeprazole must be protected from contact with acidic gastrointestinal juice by an enteric-coating to maintain its pharmaceutical activity and describes an enteric-coated omeprazole preparation containing one or more subcoats between the core material and the enteric-coating. Non-enteric coated pharmaceutical compositions have also been described, which facilitate immediate release of the pharmaceutically active ingredient into the stomach and permit stomach uptake of pharmaceutical agents. Use of non-enteric coated compositions involves the administration of one or more buffering agents with an acid labile proton pump inhibitor. The buffering agent is thought to prevent substantial degradation of the acid labile pharmaceutical agent in the acidic environment of the stomach by raising the stomach pH. See, e.g., U.S. Pat. Nos. 5,840,737; 6,489,346; 6,645,998; and 6,699,885.

Proton pump inhibitors are typically prescribed for short-term treatment of active duodenal ulcers, gastrointestinal ulcers, gastroesophageal reflux disease (GERD), severe erosive esophagitis, poorly responsive symptomatic GERD, and pathological hypersecretory conditions such as Zollinger Ellison syndrome. These above-listed conditions commonly arise in healthy or critically ill patients of all ages, and may be accompanied by significant upper gastrointestinal bleeding.

It is believed that omeprazole, lansoprazole and other proton pump inhibiting agents reduce gastrointestinal acid production by inhibiting H⁺/K⁺-ATPase of the parietal cell, which is the final common pathway for gastrointestinal acid secretion. See, e.g., Fellenius et al., Substituted Benzimidazoles Inhibit Gastrointestinal Acid Secretion by Blocking H⁺/K⁺-ATPase, Nature, 290: 159-161 (1981); Wallmark et al., The Relationship Between Gastrointestinal Acid Secretion and Gastrointestinal H⁺/K⁺-ATPase Activity, J. Biol. Chem., 260: 13681-13684 (1985); and Fryklund et al., Function and Structure of Parietal Cells After H⁺/K⁺-ATPase Blockade, Am. J. Physiol., 254 (1988).

Proton pump inhibitors have the ability to act as weak bases which reach parietal cells from the blood and diffuse into the secretory canaliculi. There the drugs become protonated and thereby trapped. The protonated compound can then rearrange to form a sulfenamide which can covalently interact with sulfhydryl groups at critical sites in the extra cellular (luminal) domain of the membrane-spanning H⁺/K⁺-ATPase. See, e.g., Hardman et al., Goodman & Gilman's The Pharmacological Basis of Therapeutics, 907 (9th ed. 1996). As such, proton pump inhibitors are prodrugs that must be activated within parietal cells to be effective. The specificity of the effects of proton pump inhibiting agents is also dependent upon: (a) the selective distribution of H⁺/K⁺-ATPase; (b) the requirement for acidic conditions to catalyze generation of the reactive inhibitor; and (c) the trapping of the protonated drug and the cationic sulfenamide within the acidic canaliculi and adjacent to the target enzyme.

Prokinetic Agents

Prokinetic agents may be prescribed in the treatement of vaious gastrointestinal diseases, such as gastroesophageal reflux disease (GERD), inflammatory bowel disease, or to treat primary gastrointestinal motility disorders, such as diffuse esophageal spasm or irritable bowel syndrome. Motility disorders of the gastrointestinal tract may be caused by neural, muscular, or receptor damaage dysfunction. Examples of neural, muscular, and receptor damage or dysfunction include (but are not limited to) diabetic gastroparesis, scleroderma, or the carcinoid syndrome.

For example, motility disorders can occur, when the nerves in the gastrointestinal tract are missing, immature, or damaged, e.g., by infections or toxins. Motility disorders can also occur when the nerves are adversely influenced by chemical substances from inside the body or outside the body. Additionally, motility disorders may occur when the GI muscles are diseased—either from a genetic defect (such as some forms of muscular dystrophy) or an acquired disorder (such as, for example, progressive systemic sclerosis and amyloidosis). Of course, there are other motility disorcers for which the etiology is not known, such as irritable bowel syndrome or functional dyspepsia.

Heartburn and constipation are two of the most common symptoms of motility disorders. Other symptoms include, for example, chronic vomiting, nausea, cramping, bloating, abdominal distention and diarrhea after eating. The most common motility disturbance is termed “irritable bowel syndrome” which accounts for about 50% of all patients. Chronic intestinal pseudo-obstruction is the name given to a group of rare nerve and muscle disorders which severely affect gastrointestinal motility. Many children and adults with chronic intestinal pseudo-obstruction require tube feedings or parenteral nutrition.

Prokinetic agents would be useful in concomitant therapy with proton pump inhibitors to treat patients with GERD, erosive esophagitis or functional dyspepsia. PPI and prokinetic agent combinations increase the tone of the lower esophageal sphincter, decrease the number of transient lower esphageal relaxations, and increase gastric emptying while the proton pump inhibitor is administered which decreases the volume of gastric juice available for reflux into the esphagus and increases the pH so that refluxed gastric contents are much less injurious to the esophageal mucosa.

### SUMMARY OF THE INVENTION

Pharmaceutical compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, are provided herein. Methods are provided for treating gastric acid related disorders in a subject, using pharmaceutical compositions of the present invention.

Proton pump inhibitors include, but are not limited to, omeprazole, hydroxyomeprazole, esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, or prodrug thereof. In one embodiment, the proton pump inhibitor is omeprazole or a free base, free acid, salt, hydrate, ester, amide, enantiomer, isomer, tautomer, polymorph, or prodrug thereof. Compositions can contain between about 5 mgs to about 200 mgs of proton pump inhibitor, specifically about 5 mg, about 10 mg, about 15 mg, about 20 mg, about 30 mg, about 40 mg, about 60 mg, or about 80 mg of the proton pump inhibitor. In alternative embodiments, compositions can contain between about 250-3000 mg of proton pump inhibitor.

Prokinetic agents include but are not limited to 5-HT inhibitors such as 5-HT₃ inhibitors (e.g., ondasetron, granisetron, and dolanserton) and 5-HT₄ inhibitors (e.g., cisapride), bulk forming agents such as phylium, polycarbophil, and fiber; intraluminal agents such as bismuth; antimotility agents such as loperamide and clonidine; saline laxatives; and luminally active osmotic agents such as magnesium sulfate and sodium phosphate. Other exemplary prokinetic agents include mosapride, metoclopramide, domperidone, clebopride, erythromycin (e.g., erythromycin ethylsuccinate and erythromycin lactobionate), bethanechol and bethanechol chloride, norcisapride, and neostigmine.

Compositions that include (a) a therapeutically effective amount of omeprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₃ receptor, are provided herein.

Compositions that include (a) a therapeutically effective amount of omeprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₄ receptor, are provided herein.

Compositions that include (a) a therapeutically effective amount of omeprazole, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent selected from ondansetron, granisetron, dolanserton, cisapride, norcisapride, loperamide, clonidine, metaclopramide, domperidone, mosapride, itopride, levopride, tiropramide, clebopride, dropreidol, promethazine, prochlorperazine, erythromycin ethylsuccinate, erythromycin lactobionate, bethanechol, bethanechol chloride, norcisapride, and neostigmine, are provided herein.

Compositions that include (a) a therapeutically effective amount of lansoprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₃ receptor, are provided herein.

Compositions that include (a) a therapeutically effective amount of lansoprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₄ receptor, are provided herein.

Compositions that include (a) a therapeutically effective amount of lansoprazole, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent selected from ondansetron, granisetron, dolanserton, cisapride, norcisapride, loperamide, clonidine, metaclopramide, domperidone, mosapride, itopride, levopride, tiropramide, clebopride, dropreidol, promethazine, prochlorperazine, erythromycin ethylsuccinate, erythromycin lactobionate, bethanechol, bethanechol chloride, norcisapride, and neostigrnine, are provided herein.

Compositions that include (a) a therapeutically effective amount of s-omeprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₃ receptor, are provided herein.

Compositions that include (a) a therapeutically effective amount of s-omeprazole, (b) at least one buffering agent in an amount sufficient to increase gastric fluid pH to a pH that prevents acid degradation of at least some of the proton pump inhibitor in the gastric fluid, and (c) a therapeutically effective amount of a 5-HT₄ receptor, are provided herein.

Compositions that include (a) a therapeutically effective amount of s-omeprazole, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent selected from ondansetron, granisetron, dolanserton, cisapride, norcisapride, loperamide, clonidine, metaclopramide, domperidone, mosapride, itopride, levopride, tiropramide, clebopride, dropreidol, promethazine, prochlorperazine, erythromycin ethylsuccinate, erythromycin lactobionate, bethanechol, bethanechol chloride, norcisapride, and neostigmine, are provided herein.

Compositions are provided such that an initial serum concentration of the proton pump inhibitor is greater than about 100 ng/ml at any time within about 30 minutes after administering the formulation. Initial serum concentration of the proton pump inhibitor can be greater than about 100 ng/ml at any time within about 15 minutes. Initial serum concentration of the proton pump inhibitor can be greater than about 200 ng/ml at any time within about 1 hour after administration, greater than about 300 ng/ml at any time within about 45 minutes after administration.

Compositions are provided such that a serum concentration of greater than about 0.1 μg/ml can be maintained from at least about 30 minutes to about 1 hour after administration of the composition. Compositions are provided such that a serum concentration of proton pump inhibitor greater than about 100 ng/ml can be maintained from at least about 15 minutes to about 30 minutes. Compositions are provided such that a serum concentration of greater than about 100 ng/ml can be maintained from at least about 30 minutes to about 45 minutes. Compositions are provided such that a serum concentration of greater than about 250 ng/ml can be maintained from at least about 30 minutes to about 1 hour. Compositions are provided such that a serum concentration of greater than about 250 ng/ml can be maintained from at least about 30 minutes to about 45 minutes. Compositions are provided such that a serum concentration of greater than about 250 ng/ml can be maintained from at least about 15 minutes to about 30 minutes.

Compositions of the invention can be administered in an amount to maintain a serum concentration of the proton pump inhibitor greater than about 150 ng/ml from about 15 minutes to about 1 hour after administration. Compositions of the invention can be administered in an amount to maintain a serum concentration of the proton pump inhibitor greater than about 150 ng/ml from about 15 minutes to about 1.5 hours after administration. Compositions of the invention can be administered in an amount to maintain a serum concentration of the proton pump inhibitor greater than about 100 ng/ml from about 15 minutes to about 1.5 hours after administration. Compositions of the invention can be administered in an amount to maintain a serum concentration of the proton pump inhibitor greater than about 150 ng/ml from about 15 minutes to about 30 minutes after administration.

Compositions of the invention can be administered in an amount to achieve an initial serum concentration of the proton pump inhibitor greater than about 150 ng/ml at any time from about 5 mintues to about 30 minutes after administration. Compositions of the invention can be administered in an amount to achieve an initial serum concentration of the proton pump inhibitor greater than about 150 ng/ml at any time within about 30 minutes after administration.

Compositions are provided wherein, upon oral administration to the subject, the composition provides a pharmacokinetic profile such that at least about 50% of total area under serum concentration time curve (AUC) for the proton pump inhibitor occurs within about 2 hours after administration of a single dose of the composition to the subject. Compositions are provided wherein, upon oral administration to the subject, the area under the serum concentration time curve (AUC) for the proton pump inhibitor in the first 2 hours is at least about 60% of the total area. Compositions are provided wherein the area under the serum concentration time curve (AUC) for the proton pump inhibitor in the first 2 hours is at least about 70% of the total area.

Compositions are provided wherein at least about 50% of total area under the serum concentration time curve (AUC) for the proton pump inhibitor occurs within about 1.75 hours after administration of a single dose of the composition to the subject. Compositions are provided wherein at least about 50% of total area under the serum concentration time curve (AUC) for the proton pump inhibitor occurs within about 1.5 hours after administration of a single dose of the composition to the subject. Compositions are provided wherein at least about 50% of total area under the serum concentration time curve (AUC) for the proton pump inhibitor occurs within about 1 hour after administration of a single dose of the composition to the subject.

Compositions are provided wherein, upon oral administration to the subject, the composition provides a pharmacokinetic profile such that the proton pump inhibitor reaches a maximum serum concentration within about 1 hour after administration of a single dose of the composition. Compositions are provided wherein the maximum serum concentration is reached within about 45 minutes after administration of the composition. Compositions are provided wherein the maximum serum concentration is reached within about 30 minutes after administration of the composition.

Compositions are provided wherein at least some of the proton pump inhibitor is microencapsulated with a material that enhances the shelf-life of the pharmaceutical composition. Compositons are provided wherein at least some of the prokinetic agent is microencapsulated with a material that enhances the shelf-life of the pharmaceutical composition. Compositions are provided wherein some of the proton pump inhibitor and some of the prokinetic agent are microencapsulated with a material that enhances the shelf-life of the pharmaceutical composition. Materials that enhance the shelf-life of the pharmaceutical composition include, but are not limited to, cellulose hydroxypropyl ethers, low-substituted hydroxypropyl ethers, cellulose hydroxypropyl methyl ethers, methylcellulose polymers, ethylcelluloses and mixtures thereof, polyvinyl alcohol, hydroxyethylcelluloses, carboxymethylcelluloses, salts of carboxymethylcelluloses, polyvinyl alcohol, polyethylene glycol co-polymers, monoglycerides, triglycerides, polyethylene glycols, modified food starch, acrylic polymers, mixtures of acrylic polymers with cellulose ethers, cellulose acetate phthalate, sepifilms, cyclodextrins; and mixtures thereof. The cellulose hydroxypropyl ether can be, but is not limited to, Klucel® or Nisso HPC. The cellulose hydroxypropyl methyl ether can be, but is not limited to, Seppifilm-LC, Pharmacoat®, Metolose SR, Opadry YS, PrimaFlo, BenecelMP824, or BenecelMP843. The mixture of methylcellulose and hydroxypropyl and methylcellulose polymers can be, but is not limited to, Methocel®, Benecel-MC, or Metolose®. The ethylcellulose or mixture thereof can be, but are not limited to, Ethocel®, BenecelMO43, Celacal, Cumibak N.C., and E461. The polyvinyl alcohol can be, but is not limited to, Opadry AMB. The acrylic polymers or mixtures thereof include, but are not limited to, Eudragits® EPO, Eudragits® RD100, and Eudragits® E100. Other materials that enhance the shelf-life of the pharmaceutical composition include, but are not limited to, Natrosol®, Aqualon®-CMC, and Kollicoat IR®. The material that enhances the shelf-life of the pharmaceutical composition can further include other compatible materials such as an antioxidant, a plasticizer, a buffering agent, and mixtures thereof.

Compositions are provided that include (a) a therapeutically effective amount of at least one acid labile proton pump inhibitor wherein at least some of the proton pump inhibitor, (b) at least one buffering agent in an amount sufficient to increase gastric fluid pH to a pH that prevents acid degradation of at least some of the proton pump inhibitor in the gastric fluid, (c) a therapeutically effective amount of at least one prokinetic agent, and (d) at least one thickening agent, wherein the dosage form is a powder for suspension. In some embodiments, the powder for suspension is substantially uniform or creates a substantially uniform suspension when mixed.

Compositions are provided that include (a) a therapeutically effective amount of at least one acid labile proton pump inhibitor wherein at least some of the proton pump inhibitor is microencapsulated, (b) at least one buffering agent in an amount sufficient to increase gastric fluid pH to a pH that prevents acid degradation of at least some of the proton pump inhibitor in the gastric fluid, (c) a therapeutically effective amount of at least one prokinetic agent, and (d) at least one thickening agent, wherein the dosage form is a powder for suspension. In some embodiments, the powder for suspension is substantially uniform or creates a substantially uniform suspension when mixed.

Compositions are provided that include (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, (c) a therapeutically effective amount of at least one prokinetic agent wherein at least some of the prokinetic agent is coated, and (d) at least one thickening agent, wherein the dosage form is a powder for suspension. In some embodiments, the powder for suspension is substantially uniform.

Compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the compositions are free of sucralfate are provided herein.

Compositions are provided that include (a) a therapeutically effective amount of at least one acid labile proton pump inhibitor wherein at least some of the proton pump inhibitor is coated, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the proton pump inhibitor is useful for treating a gastric acid related disorder.

Compositions are provided that include (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the prokinetic agent is a 5-HT inhibitor. Compositions are provided herein wherein the 5-HT inhibitor is a 5-HT₃ or 5-HT₄ inhibitor.

Compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the buffering agent is an alkaline earth metal salt or a Group IA metal selected from a bicarbonate salt of a Group LA metal, a carbonate salt of a Group IA metal. The buffering agent can be, but is not limited to, an amino acid, an alkali metal salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof. In particular, the buffering agent can be sodium bicarbonate, sodium carbonate, calcium carbonate, magnesium oxide, magnesium hydroxide, magnesium carbonate, aluminum hydroxide, and mixtures thereof.

Compositions are provided as described herein, where the buffering agent to proton pump inhibitor ratio is at least 10:1; at least 12:1; at least 15:1; at least 20:1; at least 22:1; at least 25:1; at least 30:1; at least 35:1; and at least 40:1.

Compositions are provided as described herein, where the buffering agent is sodium bicarbonate and is present in about 0.1 mEq/mg proton pump inhibitor to about 5 mEq/mg proton pump inhibitor. Compositions are provided as described herein, where the buffering agent is a mixture of sodium bicarbonate and magnesium hydroxide, and each buffering agent is present in about 0.1 mEq/mg proton pump inhibitor to about 5 mEq/mg proton pump inhibitor. Compositions are provided as described herein, where the buffering agent is a mixture of sodium bicarbonate, calcium carbonate, and magnesium hydroxide, and each buffering agent is present in about 0.1 mEq/mg proton pump inhibitor to about 5 mEq/mg of the proton pump inhibitor.

Compositions are provided as described herein, where the buffering agent is present in an amount of about 0.1 mEq/mg to about 5 mEq/mg of the proton pump inhibitor, or about 0.25 mEq/mg to about 3 mEq/mg of the proton pump inhibitor, or about 0.3 mEq/mg to about 2.5 mEq/mg of the proton pump inhibitor, or about 0.4 mEq/mg to about 2.0 mEq/mg of the proton pump inhibitor, or about 0.5 mEq/mg to about 1.5 mEq/mg of the proton pump inhibitor. Compositions are provided as described herein, where the buffering agent is present in an amount of at least 0.25 mEq/mg to about 2.5 mEq/mg of the proton pump inhibitor, or at least about 0.4 mEq/mg of the proton pump inhibitor. Compositions are provided as described herein, where the composition includes about 200 to 3000 mg of buffering agent, or about 500 to about 2500 mg of buffering agent, or about 1000 to about 2000 mg of buffering agent, or about 1500 to about 2000 mg of buffering agent.

Compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent are provided, wherein at least some of the prokinetic agent is coated. Sutiable coatings include, but are not limited to, gastric resistant coatings such as enteric coatings, controlled-release coatings, enzymatic-controlled coatings, film coatings, sustained-release coatings, immediate-release coatings, and delayed-release coatings.

Compositions including (a) a therapeutically effecive amount of at least one acid labile proton pump inhibitor, (b) at least one buffering agent selected from sodium bicarbonate, calcium carbonate, and magnesium hydroxide, wherein the buffering agent is present in an amount sufficient to increase gastric fluid, and (c) a therapeutically effective amount of at least one prokinetic agent are provided.

Compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the composition is in a dosage form selected from a powder, a tablet, a bite-disintegration tablet, a chewable tablet, a capsule, an effervescent powder, a rapid-disintegration tablet, or an aqueous suspension produced from powder.

Compositions including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the composition is in the form of a tablet and the tablet consists of a first and a second layer where the first layer comprises at least some of the prokinetic agent and the second layer comprises at least some of the proton pump inhibitor and the buffering agent.

Compositions are provided as described herein, further including one or more excipients including, but not limited to, parietal cell activators, erosion facilitators, flavoring agents, sweetening agents, diffusion facilitators, antioxidants and carrier materials selected from binders, suspending agents, disintegration agents, filling agents, surfactants, solubilizers, stabilizers, lubricants, wetting agents, diluents, anti-adherents, and antifoaming agents.

Methods are provided for treating a gastric acid related disorder by administering to the subject a pharmaceutical composition including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the proton pump inhibitor treats the gastric acid related disorder. Methods are provided wherein the composition as described herein is formulated for stomach delivery of at least some of the proton pump inhibitor. Methods are provided wherein the composition as described herein is formulated for duodenal delivery of some of the proton pump inhibitor.

Methods are provided for treating a gastric acid related disorder by administering to a horse a pharmaceutical composition including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent.

Methods are provided for treating a gastric acid related disorder including, but not limited to duodenal ulcer disease, gastric ulcer disease, gastroesophageal reflux disease, erosive esophagitis, poorly responsive symptomatic gastroesophageal reflux disease, pathological gastrointestinal hypersecretory disease, Zollinger Ellison syndrome, heartburn, esophageal disorder, and acid dyspepsia. Method are provided wherein the proton pump inhibitor treats an episode of gastric acid related disorder. Methods are provided wherein the pharmaceutical composition prevents or treats an NSAID induced gastric acid related disorder.

Methods are provided for treating a gastric acid related disorder by administering to a subject a pharmaceutical composition including (a) a therapeutically effective amount of at least one acid labile proton pump inhibitor, (b) at least one buffering agent in an amount sufficient to increase gastric fluid pH to a pH that prevents acid degradation of at least some of the proton pump inhibitor in the gastric fluid, and (c) a therapeutically effective amount of at least one prokinetic agent, wherein the composition is in a dosage form including, but not limited to, a powder, a powder for suspension, a tablet, a caplet, a bite-disintegration tablet, a chewable tablet, a capsule, an effervescent powder, a rapid-disintegration tablet, or an aqueous suspension produced from powder.

Methods are provided wherein the composition further comprises one or more excipients including, but not limited to, parietal cell activators, erosion facilitators, flavoring agents, sweetening agents, diffusion facilitators, antioxidants and carrier materials selected from binders, suspending agents, disintegration agents, filling agents, surfactants, solubilizers, stabilizers, lubricants, wetting agents, diluents, anti-adherents, and antifoaming agents.

### DETAILED DESCRIPTION OF THE INVENTION

The present invention is directed to pharmaceutical compositions comprising a proton pump inhibitor, a buffering agent, and a prokinetic agent, wherein the compositions are useful for the treatment of a disease, condition or disorder, wherein treatment includes treating the symptoms of the disease, condition or disorder. Methods of treatment using the pharmaceutical compositions of the present invention are also described.

It has been discovered that pharmaceutical compositions comprising (1) an acid labile proton pump inhibitor, together with (2) one or more buffering agents, and (3) a prokinetic agent, provide improved relief from gastric acid related disorders.

It has been discovered that pharmaceutical compositions comprising (1) an acid labile proton pump inhibitor which is microencapsulated with a material that enhances the shelf-life of the pharmaceutical composition, together with (2) one or more buffering agents, and (3) a prokinetic agent, provide superior performance by enhancing shelf-life stability of the pharmaceutical composition during manufacturing and storage.

It has been discovered that pharmaceutical compositions comprising (1) an acid labile proton pump inhibitor, together with (2) one or more buffering agents, and (3) a prokinetic agent which is coated provide superior performance by enhancing shelf-life stability of the pharmaceutical composition during manufacture and storage.

Glossary of Terms

To more readily facilitate an understanding of the invention and its preferred embodiments, the meanings of terms used herein will become apparent from the context of this specification in view of common usage of various terms and the explicit definitions of other terms provided in the glossary below or in the ensuing description.

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e., plus or minus 0.1% to 10%, which are also effective and safe. Such dosages are thus encompassed by the scope of the claims reciting the terms “about” and “approximately.”

The phrase “acid-labile pharmaceutical agent” refers to any pharmacologically active drug subject to acid catalyzed degradation.

“Anti-adherents,” “glidants,” or “anti-adhesion” agents prevent components of the formulation from aggregating or sticking and improve flow characteristics of a material. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®)and the like.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, e.g., butylated hydroxytoluene (BHT), sodium ascorbate, and tocopherol.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone®CL, Kollidon® CL, Polyplasdone® XL-10), larch arabogalactan, Veegum®, polyethylene glycol, waxes, sodium alginate, and the like.

“Bioavailability” refers to the extent to which an active moiety, e.g., drug, prodrug, or metabolite, is absorbed into the general circulation and becomes available at the site of drug action in the body.

“Carrier materials” include any commonly used excipients in pharmaceutics and should be selected on the basis of compatibility with the proton pump inhibitor and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Character notes” include, e.g., aromatics, basis tastes, and feeling factors. The intensity of the character note can be scaled from 0-none, 1-slight, 2-moderate, or 3-strong.

A “derivative” is a compound that is produced from another compound of similar structure by the replacement of substitution of an atom, molecule or group by another suitable atom, molecule or group. For example, one or more hydrogen atom of a compound may be substituted by one or more alkyl, acyl, amino, hydroxyl, halo, haloalkyl, aryl, heteroaryl, cycloaolkyl, heterocycloalkyl, or heteroalkyl group to produce a derivative of that compound.

“Diffusion facilitators” and “dispersing agents” include materials that control the diffusion of an aqueous fluid through a coating. Exemplary diffusion facilitators/dispersing agents include, e.g., hydrophilic polymers, electrolytes, Tween® 60 or 80, PEG and the like. Combinations of one or more erosion facilitator with one or more diffusion facilitator can also be used in the present invention.

“Diluents” increase bulk of the composition to facilitate compression. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); mannitol; hydroxypropylmethylsellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” includes both the dissolution and dispersion of the dosage form when contacted with gastrointestinal fluid.

“Disintegration agents” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

“Drug absorption” or “absorption” refers to the process of movement from the site of administration of a drug toward the systemic circulation, e.g., into the bloodstream of a subject.

An “enteric coating” is a substance that remains substantially intact in the stomach but dissolves and releases the drug once the small intestine is reached. Generally, the enteric coating comprises a polymeric material that prevents release in the low pH environment of the stomach but that ionizes at a slightly higer pH, typically a pH of 4 or 5, and thus dissolves sufficiently in the small intestines to gradually release the active agent therein.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose; dextrates; dextran, starches, pregelatinized starch, sucrose, xylitol, lactitol, mannitol, sorbitol, sodium chloride, polyethylene glycol, and the like.

“Flavoring agents” or “sweeteners” useful in the pharmaceutical compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Gastrointestinal fluid” is the fluid of stomach secretions of a subject or the saliva of a subject after oral administration of a composition of the present invention, or the equivalent thereof. An “equivalent of stomach secretion” includes, e.g., an in vitro fluid having similar content and/or pH as stomach secretions such as a 1% sodium dodecyl sulfate solution or 0.1N HCl solution in water.

“Half-life” refers to the time required for the plasma drug concentration or the amount in the body to decrease by 50% from its maximum concentration.

“Lubricants” are compounds which prevent, reduce or inhibit adhesion or friction of materials. Exemplary lubricants include, e.g., stearic acid; calcium hydroxide; talc; sodium stearyl fumerate; a hydrocarbon such as mineral oil, or hydrogenated vegetable oil such as hydrogenated soybean oil (Sterotex®); higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol or a methoxypolyethylene glycol such as Carbowax™, sodium oleate, glyceryl behenate, polyethylene glycol, magnesium or sodium lauryl sulfate, colloidal silica such as Syloid™, Carb-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or l of blood serum, of a therapeutic agent that is absorbed into the bloodstream after administration. One of ordinary skill in the art would be able to measure the serum concentration or plasma concentration of a proton pump inhibitor or a prokinetic agent. See, e.g., Gonzalez H. et al., J. Chromatogr. B. Analyt. Technol. Biomed. Life Sci., vol. 780, pp 459-65, (Nov. 25, 2002).

“Parietal cell activators” or “activators” stimulate the parietal cells and enhance the pharmaceutical activity of the proton pump inhibitor. Parietal cell activators include, e.g., chocolate; alkaline substances such as sodium bicarbonate; calcium such as calcium carbonate, calcium gluconate, calcium hydroxide, calcium acetate and calcium glycerophosphate; peppermint oil; spearmint oil; coffee; tea and colas (even if decaffeinated); caffeine; theophylline; theobromine; amino acids (particularly aromatic amino acids such as phenylalanine and tryptophan); and combinations thereof.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Plasma concentration” refers to the concentration of a substance in blood plasma or blood serum of a subject. It is understood that the plasma concentration of a therapeutic agent may vary many-fold between subjects, due to variability with respect to metabolism of therapeutic agents. In accordance with one aspect of the present invention, the plasma concentration of a proton pump inhibitors and/or prokinetic agent may vary from subject to subject. Likewise, values such as maximum plasma concentraton (Cₘₐₓ) or time to reach maximum serum concentration (Tₘₐₓ), or area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean plasma concentrations are disclosed for a population of subjects, these mean values may include substantial variation.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, and triacetin.

“Prevent” or “prevention” when used in the context of a gastric acid related disorder means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease.

A “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug which renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J Physio.l, 269:G210-218 (1995); McLoed et al., Gastroenterol., 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987.

“Serum concentration” refers to the concentration of a substance such as a therapeutic agent, in blood plasma or blood serum of a subject. It is understood that the serum concentration of a therapeutic agent may vary many-fold between subjects, due to variability with respect to metabolism of therapeutic agents. In accordance with one aspect of the present invention, the serum concentration of a proton pump inhibitors and/or prokinetic agent may vary from subject to subject. Likewise, values such as maximum serum concentraton (Cₘₐₓ) or time to reach maximum serum concentration (Tₘₐₓ), or total area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean serum concentrations are disclosed for a population of subjects, these mean values may include substantial variation.

“Solubilizers” include compounds such as citric acid, succinic acid, fumaric acid, malic acid, tartaric acid, maleic acid, glutaric acid, sodium bicarbonate, sodium carbonate and the like.

“Stabilizers” include compounds such as any antioxidation agents, buffers, acids, and the like.

“Suspending agents” or “thickening agents” include compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium carboxymethylcellulose; methylcellulose; hydroxy-propylmethylcellulose; polysorbate-80, hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone and the like.

“Surfactants” include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF); and the like.

A “therapeutically effective amount” or “effective amount” is that amount of a pharmaceutical agent to achieve a pharmacological effect. The term “therapeutically effective amount” includes, for example, a prophylactically effective amount. An “effective amount” of a proton pump inhibitor is an amount effective to achieve a desired pharmacologic effect or therapeutic improvement without undue adverse side effects. For example, an effective amount of a proton pump inhibitor refers to an amount of proton pump inhibitor that reduces acid secretion, or raises gastrointestinal fluid pH, or reduces gastrointestinal bleeding, or reduces the need for blood transfusion, or improves survival rate, or provides for a more rapid recovery from a gastric acid related disorder. An “effective amount” of a prokinetic agent is an amount effective to achieve a desired pharmacological effect on the subject's condition, without undue adverse side effects. The effective amount of a pharmaceutical agent will be selected by those skilled in the art depending on the particular patient and the disease level. It is understood that “an effect amount” or “a therapeutically effective amount” can vary from subject to subject, due to variation in metabolism of therapeutic agents such as proton pump inbhibitors and/or prokinetic agents, age, weight, general condition of the subject, the condition being treated, the severity of the condition being treated, and the judgment of the prescribing physician.

“Total intensity of aroma” is the overall immediate impression of the strength of the aroma and includes both aromatics and nose feel sensations.

“Total intensity of flavor” is the overall immediate impression of the strength of the flavor including aromatics, basic tastes and mouth feel sensations.

“Treat” or “treatment” as used in the context of a gastric acid related disorder refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. “Treat” or “treatment” as used in the context of a prokinetic agent refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, and the like.

Combination Therapy

Compositions and methods for combination therapy are provided herein. In accordance with one aspect, the pharmaceutical compositions disclosed herein are used to treat a gastric acid related disorder. In one embodiment, pharmaceutical compositions disclosed herein are used treat a subject suffering from a gastric acid related disorder.

Combination therapies contemplated by the present invention can be used as part of a specific treatment regimen intended to provide a beneficial effect from the co-action of the proton pump inhibitor and the prokinetic agent.

It is understood that the dosage regimen to treat, prevent, or ameliorate the condition(s) for which relief is sought, can be modified in accordance with a variety of factors. These factors include the type of gastric acid disorder from which the subject suffers, the proton pump inhibitor being administered, the prokinetic agent being administered, as well as the age, weight, sex, diet, and medical condition of the subject. Thus, the dosage regimen actually employed can vary widely and therefore can deviate from the dosage regimens set forth herein.

In accordance with one aspect, compositions and methods of the present invention are designed to produce release of the proton pump inhibitor to the site of delivery, while substantially preventing or inhibiting acid degradation of the proton pump inhibitor. The present invention includes compositions and methods for treating, preventing, reversing, halting or slowing the progression of a gastric acid related disorder once it becomes clinically evident, or treating the symptoms associated with or related to the gastric acid related disorder, by administering to the subject a composition of the present invention. The subject may already have a gastric acid related disorder at the time of administration, or be at risk of developing a gastric acid related disorder. The symptoms or conditions of a gastric acid related disorder in a subject can be determined by one skilled in the art and are described in standard textbooks. The method comprises the oral administration of an effective amount of one or more compositions of the present invention to a subject in need thereof. Gastric acid related disorders suitable for treatment using compositions and methods of the present invention include, but are not limited to, duodenal ulcer disease, gastrointestinal ulcer disease, gastroesophageal reflux disease (GERD), erosive esophagitis, poorly responsive symptomatic gastroesophageal reflux disease, pathological gastrointestinal hypersecretory disease, Zollinger Ellison Syndrome, heartburn, esophageal disorder, and acid dyspepsia.

As disclosed herein, proton pump inhibitors and/or prokinetic agents can be formulated to deliver rapid relief as well as sustained relief of a gastric acid related disorder. According to the methods of the invention, the formulation of the proton pump inhibitor is chosen on the basis of the type of gastric acid related disorder suffered by the subject.

In one embodiment, a subject is administered a composition containing a proton pump inhibitor formulated to give rapid relief for an episode of a gastric acid related disorder. In another embodiment, a subject is administered a composition including uncoated proton pump inhibitor formulated to provide rapid relief and coated proton pump inhibitor to prevent or treat recurring episodes of the gastric acid related disorder, where the composition also contains a prokinetic agent. In another aspect of the invention, a subject is administered a composition containing a proton pump inhibitor and a prokinetic agent, wherein at least some of the prokinetic agent is coated. In yet another aspect of the invention, a subject is administered a composition containing a proton pump inhibitor and a prokinetic agent, wherein at least some of the prokinetic agent is coated with an immediate release coating for improved shelf-life of the pharmaceutical composition. According to another aspect of the invention, a subject is administered a composition containing a proton pump inhibitor and a prokinetic agent, wherein at least some of the prokinetic agent is coated with an enteric coating which is designed for a delayed release of the prokinetic agent.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

Proton Pump Inhibitors

The terms “proton pump inhibitor,” “PPI,” and “proton pump inhibiting agent” can be used interchangeably to describe any acid labile pharmaceutical agent possessing pharmacological activity as an inhibitor of H⁺/K⁺-ATPase. A proton pump inhibitor may, if desired, be in the form of free base, free acid, salt, ester, hydrate, anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active.

In various embodiments, the proton pump inhibitor can be a substituted bicyclic aryl-imidazole, wherein the aryl group can be, e.g., a pyridine, a phenyl, or a pyrimidine group and is attached to the 4 - and 5-positions of the imidazole ring. Proton pump inhibitors comprising a substituted bicyclic aryl-imidazoles include, but are not limited to, omeprazole, hydroxyomeprazole, esomeprazole, lansoprazole, pantoprazole, rabeprazole, dontoprazole, habeprazole, perprazole, tenatoprazole, ransoprazole, pariprazole, leminoprazole, or a free base, free acid, salt, hydrate, ester, amide, enantiomer, isomer, tautomer, polymorph, prodrug, or derivative thereof. See, e.g., The Merck Index, Merck & Co. Rahway, N.J. (2001).

Other proton pump inhibitors include but are not limited to: soraprazan (Altana); ilaprazole (U.S. Pat. No. 5,703,097) (Il-Yang); AZD-0865 (AstraZeneca); YH-1885 (PCT Publication WO 96/05177) (SB-641257) (2-pyrimidinamine, 4-(3,4-dihydro-1-methyl-2(1H)-isoquinolinyl)-N-(4-fluorophenyl)-5,6-dimethyl-monohydrochloride)(YuHan); BY-112 (Altana); SPI-447 (Imidazo(1,2-a)thieno(3,2-c)pyridin-3-amine,5-methyl-2-(2-methyl-3-thienyl) (Shinnippon); 3-hydroxymethyl-2-methyl-9-phenyl-7H-8,9-dihydro-pyrano(2,3-c)-imidazo(1,2-a)pyridine (PCT Publication WO 95/27714) (AstraZeneca); Pharmaprojects No. 4950 (3-hydroxymethyl-2-methyl-9-phenyl-7H-8,9-dihydro-pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (Toa Eiyo); HN-11203 (Nycomed Pharma); OPC-22575; pumilacidin A (BMS); saviprazole (EP 234485) (Aventis); SKandF-95601 (GSK, discontinued); Pharmaprojects No. 2522 (EP 204215) (Pfizer); S-3337 (Aventis); RS-13232A (Roche); AU-1363 (Merck); SKandF-96067 (EP 259174) (Altana); SUN 8176 (Daiichi Phama); Ro-18-5362 (Roche); ufiprazole (EP 74341) (AstraZeneca); and Bay-p-1455 (Bayer); or a free base, free acid, salt, hydrate, ester, amide, enantiomer, isomer, tautomer, polymorph, prodrug, or derivative of these compounds.

Still other proton pump inhibitors contemplated by the present invention include those described in the following U.S. Pat. Nos: 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994; 6,479,075; 6,559,167.

Other substituted bicyclic aryl-imidazole compounds as well as their salts, hydrates, esters, amides, enantiomers, isomers, tautomers, polymorphs, prodrugs, and derivatives may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable salts,” or “salts,” include, e.g., the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

In one embodiment, acid addition salts are prepared from the free base using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In other embodiments, an acid addition salt is reconverted to the free base by treatment with a suitable base. In a further embodiment, the acid addition salts of the proton pump inhibitors are halide salts, which are prepared using hydrochloric or hydrobromic acids. In still other embodiments, the basic salts are alkali metal salts, e.g., sodium salt.

Salt forms of proton pump inhibiting agents include, but are not limite to: a sodium salt form such as esomeprazole sodium, omeprazole sodium, rabeprazole sodium, pantoprazole sodium; or a magnesium salt form such as esomeprazole magnesium or omeprazole magnesium, described in U.S. Pat. No. 5,900,424; a calcium salt form; or a potassium salt form such as the potassium salt of esomeprazole, described in U.S. patent application. Ser. No. 02/0198239 and U.S. Pat. No. 6,511,996. Other salts of esomeprazole are described in U.S. Pat. No. 4,738,974 and U.S. Pat. No. 6,369,085. Salt forms of pantoprazole and lansoprazole are discussed in U.S. Pat. Nos. 4,758,579 and 4,628,098, respectively.

In one embodiment, preparation of esters involves functionalization of hydroxyl and/or carboxyl groups which may be present within the molecular structure of the drug. In one embodiment, the esters are acyl-substituted derivatives of free alcohol groups, e.g., moieties derived from carboxylic acids of the formula RCOOR₁ where R₁ is a lower alkyl group. Esters can be reconverted to the free acids, if desired, by using conventional procedures such as hydrogenolysis or hydrolysis.

“Amides” may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole such as those described in U.S. Pat. Nos.: 6,262,085; 6,262,086; 6,268,385; 6,312,723; 6,316,020; 6,326,384; 6,369,087; and 6,444,689; and U.S. patent Publication No. 02/0156103.

An exemplary “isomer” of a substituted bicyclic aryl-imidazole is the isomer of omeprazole including but not limited to isomers described in: Oishi et al., Acta Cryst. (1989), C45, 1921-1923; U.S. Pat. No. 6,150,380; U.S. patent Publication No. 02/0156284; and PCT Publication No. WO 02/085889.

Exemplary “polymorphs” include, but are not limited to, those described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

Micronized Proton Pump Inhibitor

Particle size of the proton pump inhibitor can affect the solid dosage form in numerous ways. Since decreased particle size increases in surface area (S), the particle size reduction provides an increase in the rate of dissolution (dM/dt) as expressed in the Noyes-Whitney equation below:
dM/dt=dS/h(Cs−C)
M=mass of drug dissolved; t=time; D=diffusion coefficient of drug; S=effective surface area of drug particles; H=stationary layer thickness; Cs=concentration of solution at saturation; and C=concentration of solution at time t.

Because omeprazole, as well as other proton pump inhibitors, has poor water solubility, to aid the rapid absorption of the drug product, various embodiments of the present invention use micronized proton pump inhibitor is used in the drug product formulation.

In some embodiments, the average particle size of at least about 90% the micronized proton pump inhibitor is less than about 40 μm, or less than about 35 μm, or less than about 30 μm, or less than about 25 μm, or less than about 20 μm, or less than about 15 μm, or less than about 10 μm. In other embodiments, at least 80% of the micronized proton pump inhibitor has an average particle size of less than about 40 μm, or less than about 35 μm, or less than about 30 μm, or less than about 25 μm, or less than about 20 μm, or less than about 15 μm, or less than about 10 μm. In still other embodiments, at least 70% of the micronized proton pump inhibitor has an average particle size of less than about 40 μm, or less than about 35 μm, or less than about 30 μm, or less than about 25 μm, or less than about 20 μm, or less than about 15 μm, or less than about 10 μm.

Compositions are provided wherein the micronized proton pump inhibitor is of a size which allows greater than 75% of the proton pump inhibitor to be released within about 1 hour, or within about 50 minutes, or within about 40 minutes, or within about 30 minutes, or within about 20 minutes, or within about 10 minutes or within about 5 minutes of dissoluion testing. In another embodiment of the invention, the micronized proton pump inhibitor is of a size which allows greater than 90% of the proton pump inhibitor to be released within about 1 hour, or within about 50 minutes, or within about 40 minutes, or within about 30 minutes, or within about 20 minutes, or within about 10 minutes or within about 5 minutes of dissoluion testing. See U.S. Provisional Application No. 60/488,324 filed Jul. 18, 2003, and any subsequent application claiming priority to this application, all of which are incorporated by reference in their entirety.

Buffering Agents

The pharmaceutical composition of the invention comprises one or more buffering agents. A class of buffering agents useful in the present invention include, but are not limited to, buffering agents possessing pharmacological activity as a weak base or a strong base. In one embodiment, the buffering agent, when formulated or delivered with an proton pump inhibiting agent, functions to substantially prevent or inhibit the acid degradation of the proton pump inhibitor by gastrointestinal fluid for a period of time, e.g., for a period of time sufficient to preserve the bioavailability of the proton pump inhibitor administered. The buffering agent can be delivered before, during and/or after delivery of the proton pump inhibitor. In one aspect of the present invention, the buffering agent includes a salt of a Group IA metal (alkali metal), including, e.g., a bicarbonate salt of a Group IA metal, a carbonate salt of a Group IA metal; an alkaline earth metal buffering agent (Group IIA metal); an aluminum buffering agent; a calcium buffering agent; or a magnesium buffering agent.

Other buffering agents suitable for the present invention include, e.g., alkali metal (a Group IA metal including, but not limited to, lithium, sodium, potassium, rubidium, cesium, and francium) or alkaline earth metal (Group IIA metal including, but not limited to, beryllium, magnesium, calcium, strontium, barium, radium) carbonates, phosphates, bicarbonates, citrates, borates, acetates, phthalates, tartrate, succinates and the like, such as sodium or potassium phosphate, citrate, borate, acetate, bicarbonate and carbonate.

In various embodiments, a buffering agent includes an amino acid, an alkali metal salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (See, e.g., lists provided in The Merck Index, Merck & Co. Rahway, N.J. (2001)). Certain proteins or protein hydrolysates that rapidly neutralize acids can serve as buffering agents in the present invention. Combinations of the above mentioned buffering agents can be used in the pharmaceutical compositions described herein.

The buffering agents useful in the present invention also include buffering agents or combinations of buffering agents that interact with HCl (or other acids in the environment of interest) faster than the proton pump inhibitor interacts with the same acids. When placed in a liquid phase, such as water, these buffering agents produce and maintain a pH greater than the pKa of the proton pump inhibitor.

In various embodiments, the buffering agent is selected from sodium bicarbonate, sodium carbonate, calcium carbonate, magnesium oxide, magnesium hydroxide, magnesium carbonate, aluminum hydroxide, and mixtures thereof. In another embodiment, the buffering agent is sodium bicarbonate and is present in about 0.1 mEq/mg proton pump inhibitor to about 5 mEq/mg proton pump inhibitor. In yet another embodiment, the buffering agent is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. In still another embodiment, the buffering agent is a mixture of at least two buffers selected from sodium bicarbonate, calcium carbonate, and magnesium hydroxide, wherein each buffer is present in about 0.1 mEq/mg proton pump inhibitor to about 5 mEq/mg of the proton pump inhibitor.

Compositions are provided as described herein, wherein the buffering agent is present in an amount of about 0.1 mEq/mg to about 5 mEq/mg of the proton pump inhibitor, or about 0.25 mEq/mg to about 3 mEq/mg of the proton pump inhibitor, or about 0.3 mEq/mg to about 2.5 mEq/mg of the proton pump inhibitor, or about 0.4 mEq/mg to about 2.0 mEq/mg of the proton pump inhibitor, or about 0.5 mEq/mg to about 1.5 mEq/mg of the proton pump inhibitor. Compositions are provided as described herein, wherein the buffering agent is present in an amount of at least 0.25 mEq/mg to about 2.5 mEq/mg of the proton pump inhibitor, or at least about 0.4 mEq/mg of the proton pump inhibitor.

In one aspect of the invention, compositions are provided wherein the buffering agent is present in the pharmaceutical compositions of the present invention in an amount of about 1 mEq to about 160 mEq per dose, or about 5 mEq, or about 10 mEq, or about 11 mEq, or about 15 mEq, or about 19 mEq, or about 20 mEq, or about 22 mEq, or about 23 mEq, or about 24 mEq, or about 25 mEq, or about 30 mEq, or about 31 mEq, or about 35 mEq, or about 40 mEq, or about 45 mEq, or about 50 mEq, or about 60 mEq, or about 70 mEq, or about 80 mEq, or about 90 mEq, or about 100 mEq, or about 110 mEq, or about 120 mEq, or about 130 mEq, or about 140 mEq, or about 150 mEq, or about 160 mEq per dose.

In one embodiment, the pharmaceutical composition comprises between about 5 mEq to about 20 mEq, or between about 5 mEq to about 15 mEq, or between about 5 mEq to about 12 mEq, or between about 7 mEq to about 12 mEq of buffering agent, wherein the pharmaceutical composition is substantially free from amino acids. In another embodiment, the pharmaceutical composition comprises about 5 mEq, or about 7 mEq, or about 10 mEq, or about 12 mEq, or about 15 mEq, or about 17 mEq, or about 20 mEq of buffering agent, wherein the pharmaceutical composition is substantially free from amino acids.

In another aspect of the invention, compositions are provided wherein the buffering agent is present in the composition in an amount, on a weight to weight (w/w) basis, of more than about 5 times, or more than about 10 times, or more than about 20 times, or more than about 30 times, or more than about 40 times, or more than about 50 times, or more than about 60 times, or more than about 70 times, or more than about 80 times, or more than about 90 times, or more than about 100 times the amount of the proton pump inhibiting agent.

In another aspect of the invention, compositions are provided wherein the amount of buffering agent present in the pharmaceutical composition is between 200 and 3500 mg. In some embodiments, the amount of buffering agent present in the pharmaceutical composition is about 200 mg, or about 300 mg, or about 400 mg, or about 500 mg, or about 600 mg, or about 700 mg, or about 800 mg, or about 900 mg, or about 1060 mg, or about 1100 mg, or about 1200 mg, or about 1300 mg, or about 1400 mg, or about 1500 mg, or about 1600 mg, or about 1700 mg, or about 1800 mg, or about 1900 mg, or about 2000 mg, or about 2100 mg, or about 2200 mg, or about 2300 mg, or about 2400 mg, or about 2500 mg, or about 2600 mg, or about 2700 mg, or about 2800 mg, or about 2900 mg, or about 3000 mg, or about 3200 mg, or about 3500 mg.

Prokinetic Agents

Prokinetic agents suitable for use in the present invention include but are not limited to 5-HT inhibitors such as 5-HT₃ inhibitors (e.g., ondasetron, granisetron, and dolanserton) and 5-HT₄ inhibitors (e.g., cisapride); bulk forming agents such as phylium, polycarbophil, and fiber; intraluminal agents such as bismuth; antimotility agents such as loperamide and clonidine; saline laxatives; and luminally active osmotic agents such as magnesium sulfate and sodium phosphate. Other exemplary prokinetic agents include mosapride, metoclopramide, domperidone, clebopride, erythromycin (e.g., erythromycin ethylsuccinate and erythromycin lactobionate), bethanechol and bethanechol chloride, norcisapride, and neostigmine.

Stability Enhancers

In accordance with one aspect of the present invention, compositions may include microencapsulation of one or more of: the proton pump inhibitor; the prokinetic agent; or the buffering agent, in order to enhance the shelf-life of the composition. See U.S. Provisional Application No. 60/488,321 filed Jul. 18, 2003, and any subsequent application claiming priority to it, all of which are incorporated by reference in their entirety. Materials useful for enhancing the shelf-life of the pharmaceutical compositions of the present invention include materials compatible with the proton pump inhibitor of the pharmaceutical compositions which sufficiently isolate the proton pump inhibitor from other non-compatible excipients. Materials compatible with the proton pump inhibitors of the present invention are those that enhance the shelf-life of the proton pump inhibitor, i.e., by slowing or stopping degradation of the proton pump inhibitor.

Exemplary microencapsulation materials useful for enhancing the shelf-life of pharmaceutical compositions comprising a proton pump inhibitor include, but are not limited to: cellulose hydroxypropyl ethers (HPC) such as Klucel® or Nisso HPC; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, Benecel MP824, and Benecel MP843; methylcellulose polymers such as Methocel® and Metolose®; Ethylcelluloses (EC) and mixtures thereof such as E461, Ethocel®, Aqualon®-EC, Surelease®; Polyvinyl alcohol (PVA) such as Opadry AMB; hydroxyethylcelluloses such as Natrosol®; carboxymethylcelluloses and salts of carboxymethylcelluloses (CMC) such as Aqualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of,HPMC and stearic acid, cyclodextrins; and mixtures of these materials.

In various embodiments, a buffering agent such as sodium bicarbonate is incorporated into the microencapsulation material. In other embodiments, an antioxidant such as BHT is incorporated into the microencapsulation material. In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for enhancing the shelf-life of the pharmaceutical compositions is from the USP or the National Formulary (NF).

In further embodiments, one or more other compatible materials are present in the microencapsulation material. Exemplary materials include, but are not limited to, pH modifiers, parietal cell activators, erosion facilitators, diffusion facilitators, anti-adherents, anti-foaming agents, antioxidants, flavoring agents, and carrier materials such as binders, suspending agents, disintegration agents, filling agents, surfactants, solubilizers, stabilizers, lubricants, wetting agents, and diluents.

According to one aspect of the invention, the prokinetic agent is coated. The coating may be, for example, a gastric resistant coating such as an enteric coating (See, e.g, WO91/16895 and WO91/16886), a controlled-release coating, an enzymatic-controlled coating, a film coating, a sustained-release coating, an immediate-release coating, or a delayed-release coating. According to another aspect of the invention, the coating may be useful for enhancing the stability of the pharmaceutical compositons of the present invention.

A pharmaceutical composition of the present invention may have an enhanced shelf-life stability if, e.g., the proton pump inhibitor has less than about 0.5% degradation after one month of storage at room temperature, or less than about 1% degradation after one month at room temperature, or less than about 1.5% degradation after one month of storage at room temperature, or less than about 2% degradation after one month storage at room temperature, or less than about 2.5% degradation after one month of storage at room temperature, or less than about 3% degradation after one month of storage at room temperature.

In other embodiments, a pharmaceutical composition of the present invention may have an enhanced shelf-life stability if the pharmaceutical composition contains less than about 5% total impurities after about 3 years of storage, or after about 2.5 years of storage, or about 2 years of storage, or about 1.5 years of storage, or about 1 year of storage, or after 11 months of storage, or after 10 months of storage, or after 9 months of storage, or after 8 months of storage, or after 7 months of storage, or after 6 months of storage, or after 5 months of storage, or after 4 months of storage, or after 3 months of storage, or after 2 months of storage, or after 1 month of storage.

In farther embodiments, a pharmaceutical compositions of the present invention may have an enhanced shelf-life stability if the pharmaceutical composition contains less degradation of the proton pump inhibitor than proton pump inhibitor in the same formulation where the proton pump inhibitor or prokinetic agent are not microencapsulated, or the prokinetic agent is not coated, sometimes referred to as “bare.” For example, if proton pump inhibitor in the pharmaceutical composition degrades at room temperature by more than about 2% after one month of storage and the microencapsulated or coated material degrades at room temperature by less than about 2% after one month of storage, then the proton pump inhibitor has been microencapsulated with a compatible material that enhances the shelf-life of the pharmaceutical composition, or the prokinetic agent has been coated with a compatible material that enhances the shelf-life of the pharmaceutical composition.

In some embodiments, the pharmaceutical compositions have an increased shelf-life stability of at least about 5 days at room temperature, or at least about 10 days at room temperature, or at least about 15 days at room temperature, or at least about 20 days at room temperature, or at least about 25 days at room temperature, or at least about 30 days at room temperature or at least about 2 months at room temperature, or at least about 3 months at room temperature, or at least about 4 months at room temperature, or at least about 5 months at room temperature, or at least about 6 months at room temperature, or at least about 7 months at room temperature, or at least about 8 months at room temperature or at least about 9 months at room temperature, or at least about 10 months at room temperature, or at least about 11 months at room temperature, or at least about one year at room temperature, or at least about 1.5 years at room temperature, or at least about 2 years at room temperature, or at least about 2.5 years at room temperature, or about 3 years at room temperature.

In some embodiments of the present invention, the final formulation of the pharmaceutical composition will be in the form of a tablet or caplet and at least about 50%, or at least about 55%, or at least about 60%, or at least about 65%, or at least about 70%, or at least about 75%, or at least about 80%, or at least about 85% or at least about 90%, or at least about 92%, or at least about 95%, or at least about 98%, or at least about 99% of the microspheres survive the tabletting process, wherein microspheres that have survived the manufacturing process are those which provide the desired properties described herein.

In other embodiments, the final formulation of the pharmaceutical composition is in the form of a powder for oral suspension and the microencapsulation material surrounding the proton pump inhibitor or prokinetic agent or the coating surrounding the prokinetic agent will sufficiently dissolve in water, with or without stirring, in less than 1 hour, or less than 50 minutes, or less than 40 minutes, or less than 30 minutes, or less than 25 minutes, or less than 20 minutes, or less than 15 minutes, or less than 10 minutes or less than 5 minutes, or less than 1 minute. “Sufficiently dissolves” means that at least about 50% of the encapsulation or coating material has dissolved.

In various embodiments the material useful for enhancing the shelf-life of the pharmaceutical composition sufficiently disintegrates to release the proton pump inhibitor into the gastrointestinal fluid of the stomach within less than about 1.5 hours, or within about 10 minutes, or within about 20 minutes, or within about 30 minutes, or within about 40 minutes, or within about 50 minutes, or within about 1 hour, or within about 1.25 hours, or within about 1.5 hours after exposure to the gastrointestinal fluid. Sufficiently disintegrates means that at least about 50% of the microencapsulation material has dissolved.

Taste-Masking Materials

In accordance with another aspect, compositions and methods of the present invention may include taste-masking materials to enhance the taste of the composition. Proton pump inhibitors and some prokinetic agents are inherently bitter tasting. In one embodiment of the present invention, these bitter tasting pharmaceuticals are microencapsulated with a taste-masking material. Materials useful for masking the taste of a pharmaceutical compositions include those materials capable of microencapsulating the proton pump inhibitor and/or prokinetic agent, thereby protecting the senses from its bitter taste. Taste-masking materials of the present invention provide superior pharmaceutical compositions by e.g., creating a more palatable pharmaceutical composition as compared to pharmaceutical compositions without taste-masking and/or by creating a dosage form requiring less of the traditional flavoring agents.

The “flavor leadership” criteria used to develop a palatable product include (1) immediate impact of identifying flavor, (2) rapid development of balanced, full flavor, (3) compatible mouth feel factors, (4) no “off” flavors, and (5) short aftertaste. See, e.g., Worthington, A Matter of Taste, Pharmaceutical Executive (April 2001). The pharmaceutical compositions of the present invention improve upon one or more of these criteria.

There are a number of known methods to determine the effect of a taste-masking material such as discrimination tests for testing differences between samples and for ranking a series of samples in order of a specific characteristic; scaling tests used for scoring the specific product attributes such as flavor and appearance; expert tasters used to both quantitatively and qualitatively evaluate a specific sample; affective tests for either measuring the response between two products, measuring the degree of like or dislike of a product or specific attribute, or determine the appropriateness of a specific attribute; and descriptive methods used in flavor profiling to provide objective description of a product are all methods used in the field.

Different sensory qualities of a pharmaceutical composition such as aroma, flavor, character notes, and aftertaste can be measured using tests know in the art. See, e.g., Roy et al., Modifying Bitterness: Mechanism, Ingredients, and Applications (1997). For example, aftertaste of a product can be measured by using a time vs. intensity sensory measurement. Assays have been developed to alert a processor of formulations to the bitter taste of certain substances. Using information known to one of ordinary skill in the art, one would readily be able to determine whether one or more sensory quality of a pharmaceutical composition of the present invention has been improved by the use of the taste-masking material.

Taste of a pharmaceutical composition is important for both increasing patient compliance as well as for competing with other marketed products used for similar diseases, conditions and disorders. Taste, especially bitterness, is particularly important in pharmaceutical compositions for children since, because they cannot weigh the positive outcome (getting better) against the immediate negative experience (the bitter taste in their mouth), they are more likely to refuse a drug that tastes bad. Thus, for pharmaceutical compositions for children, it becomes even more important to mask the bitter taste.

Microencapsulation of the proton pump inhibitor can (1) lower the amount of flavoring agents necessary to create a palatable product and/or (2) mask the bitter taste of the proton pump inhibitor by separating the drug from the taste receptors. Microencapsulation of the prokinetic agent can (1) lower the amount of flavoring agents necessary to create a palatable product and/or (2) mask the bitter taste of the prokinetic agent by separating the drug from the taste receptors.

Taste-masking materials include, but are not limited to: cellulose hydroxypropyl ethers (HPC) such as Klucel® or Nisswo HPC; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, Benecel MP824, and Benecel MP843; methylcellulose polymers such as Methocel® and Metolose®; ethylcelluloses (EC) and mixtures thereof such as E461, Ethocel®, Aqualon®-EC, Surelease®; polyvinyl alcohol (PVA) such as Opadry AMB; hydroxyethylcelluloses such as Natrosol®; carboxymethylcelluloses and salts of carboxymethylcelluloses (CMC) such as Aqualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials.

In other embodiments of the present invention, additional taste-masking materials contemplated are those described in U.S. Pat. Nos. 4,851,226, 5,075,114, and 5,876,759. For further examples of taste-masking materials. See, e.g., Remington: The Science and Practice of Pharmacy, Nineteenth Ed. (Easton, Pa.: Mack Publishing Company, 1995); Hoover, John E., Remington's Pharmaceutical Sciences (Mack Publishing Co., Easton, Pa. 1975); Liberman, H. A. and Lachman, L., Eds., Pharmaceutical Dosage Forms (Marcel Decker, New York, N.Y., 1980); and Pharmaceutical Dosage Forms and Drug Delivery Systems, Seventh Ed. (Lippincott Williams & Wilkins, 1999).

In various embodiments, a buffering agent such as sodium bicarbonate is incorporated into the microencapsulation material. In other embodiments, an antioxidant such as BHT is incorporated into the microencapsulation material. In yet another embodiment, sodium chloride is incorporated into the taste masking material. In still other embodiments, plasticizers such as polyethylene glycol and/or stearic acid are incorporated into the microencapsulation material.

In further embodiments, one or more other compatible materials are present in the microencapsulation material. Exemplary materials include, e.g., pH modifiers, parietal cell activators, erosion facilitators, diffusion facilitators, anti-adherents, anti-foaming agents, antioxidants, flavoring agents, and carrier materials such as binders, suspending agents, disintegration agents, filling agents, surfactants, solubilizers, stabilizers, lubricants, wetting agents, diluents.

In addition to microencapsulating the proton pump inhibitors and/or the prokinetic agent with a taste-masking material as described herein, the pharmaceutical compositions of the present invention may also comprise one or more flavoring agents.

“Flavoring agents” or “sweeteners” useful in the pharmaceutical compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In other embodiments, sodium chloride is incorporated into the pharmaceutical composition. Based on the proton pump inhibitor, buffering agent, and excipients, as well as the amounts of each one, one of skill in the art would be able to determine the best combination of flavors to provide the optimally flavored product for consumer demand and compliance. See, e.g., Roy et al., Modifying Bitterness: Mechanism, Ingredients, and Applications (1997).

In one embodiment, one or more flavoring agents are mixed with the taste-masking material prior to microencapsulating the proton pump inhibitor and/or prokinetic agent, and are therefore part of the taste-masking material. In other embodiments, the flavoring agent is mixed with non-compatible excipients during the formulation process and is therefore not in contact with the proton pump inhibitor and/or prokinetic agent, and not part of the microencapsulation material.

In another embodiment, a buffering agent, such as sodium bicarbonate, is also microencapsulated with one or more taste-masking materials.

In another embodiment, the weight fraction of the taste masking material is, e.g., about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

In other embodiments of the present invention, the amount of flavoring agent necessary to create a palatable product, as compared to a pharmaceutical composition comprising non-microencapulated proton pump inhibitor and/or the prokinetic agent, is decreased by 5% or less, or by 5% to 10%, or by 10% to 20%, or by 20% to 30%, or by 30% to 40%, or by 40% to 50%, or by 50% to 60%, or by 60% to 70%, or by 70% to 80%, or by 80% to 90%, or by 90% to 95%, or by greater than 95%. In still other embodiments, no flavoring agent is necessary to create a more palatable pharmaceutical composition as compared to a similar pharmaceutical composition comprising non-microencapulated proton pump inhibitor and/or prokinetic agent.

In various embodiments of the invention, the total amount of flavoring agent present in the pharmaceutical composition is less than 20 grams, or less than 15 grams, or less than 10 grams, or less than 8 grams, or less than 5 grams, or less than 4 grams, or less than 3.5 grams, or less than 3 grams, or less than 2.5 grams or less than 2 grams, or less than 1.5 grams, or less than 1 gram, or less than 500 mg, or less than 250 mg, or less than 150 mg, or less than 100 mg, or less than 50 mg.

Methods of Microencapsulation

The proton pump inhibitor, buffering agent, and/or prokinetic agent may be microencapsulated by methods known by one of ordinary skill in the art. Such known methods include, e.g., spray drying processes, spinning disk-solvent processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used.

The spinning disk method allows for: 1) an increased production rate due to higher feed rates and use of higher solids loading in feed solution, 2) the production of more spherical particles, 3) the production of a more even coating, and 4) limited clogging of the spray nozzle during the process.

Spray drying is often more readily available for scale-up. In various embodiments, the material used in the spray-dry encapsulation process is emulsified or dispersed into the core material in a concentrated form, e.g., 10-60% solids. The microencapsulation material is, in one embodiment, emulsified until about 1 to 3 μm droplets are obtained. Once a dispersion of proton pump inhibitor and encapsulation material are obtained, the emulsion is fed as droplets into the heated chamber of the spray drier. In some embodiments, the droplets are sprayed into the chamber or spun off a rotating disk. The microspheres are then dried in the heated chamber and fall to the bottom of the spray drying chamber where they are harvested.

In some embodiments of the present invention, the micropheres have irregular geometries. In other embodiments, the microspheres are aggregates of smaller particles.

In various embodiments, the proton pump inhibitor and/or the prokinetic agents are present in the microspheres in an amount greater than 1%, greater than 2.5%, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80%, greater than 85%, greater than 90% greater than 95% or greater than 98% weight percent of the proton pump inhibitor to the microencapsulation material used to enhance the stability of the pharmaceutical composition or the taste-masking material.

Coatings

In accordance with another aspect of the present invention, all or part of the prokinetic agent may be coated. In various embodiments contemplated by the present invention, the prokinetic agent is coated with, for example, a gastric resistant coating such as an enteric coating, a controlled-release coating, an enzymatic-controlled coating, a film coating, a sustained-release coating, an immediate-release coating, a delayed-release coating, or a moisture barrier coating. See, e.g, Remington's Pharmaceutical Sciences, 20th Edition (2000).

In accordance with another aspect of the invention, the prokinetic agent is enterically coated. Suitable enteric coating materials include, but are not limited to, polymerized gelatin, shellac, methacrylic acid copolymer type C NF, cellulose butyrate, phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxypropyl methylcellulose acetate, dioxypropyl methylcellulose succinate, carboxymethyl ethylcellulose (CMEC), hydroxypropyl methylcellulose succinate, and acrylic acid polymers and copolymers such as those formed from methyl acrylate, theyl acrylate, methyl methacrylate and/or ehtyl methacrylate with copolymers of acrylic and methacrylic acid esters (e.g., Eudragit NE, Eudragit RL, Eudragit RS). In accordance with one aspect of the present invention, all or part of the proton pump inhibitor may be coated. In various embodiments contemplated by the present invention, the proton pump inhibitor is coated with, for example, a gastric resistant coating such as an enteric coating, a controlled-release coating, an enzymatic-controlled coating, a film coating, a sustained-release coating, an immediate-release coating, a delayed-release coating, or a moisture barrier coating. See, e.g, Remington's Pharmaceutical Sciences, 20th Edition (2000).

In accordance with another aspect of the invention, either the proton pump inhibiting agent or the prokinetic agent is coated. In other aspectes of the invention, some or all of the proton pump inhibitor and some or all of the prokinetic agent are coated. In accordance with another aspect of the invention, the dosage form (such as a tablet, caplet, or capsule) is coated to aid swallowing. The proton pump inhibiting agent may be coated with the same material as used to coat the prokinetic agent or a different material. Additionally, the coating used to coat the whole dosage form (such as a film coating) may be the same as or different from the coating used to coat the proton pump inhibiting agent and/or the prokinetic agent.

Pharmaceutical compositions having multisite absorption profiles of the prokinetic agent are provided herein. In accordance with one aspect of the invention, some of the prokinetic agent is formulated for immediate release and some of the prokinetic agent is formulated for delayed release. In accordance with one aspect of the invention, the delayed release coating is an enteric coating.

Pharmaceutical compositions having multisite absorption profiles of the proton pump inhibitor are provided herein. In accordance with one aspect of the invention, some of the proton pump inhibitor is formulated for immediate release and some of the part of the proton pump inhibitor is formulated for delayed release. In accordance with one aspect of the invention, the delayed release coating is an enteric coating. In accordance with another aspect of the invention, the proton pump inhibitor is coated with a thin enteric coating.

Dosage

The pharmaceutical compositions of the present invention comprising a proton pump inhibiting agent and a prokinetic agent are administered and dosed in accordance with good medical practice, taking into account the clinical condition of the individual patient, the site and method of administration, scheduling of administration, and other factors known to medical practitioners. In human therapy, it is important to provide a dosage form that delivers the required therapeutic amount of the each therapeutic agent in vivo, and renders therapeutic agent bioavailable in a rapid manner.

Proton Pump Inhibiting Agents

The proton pump inhibiting agent is administered and dosed in accordance with good medical practice, taking into account the clinical condition of the individual patient, the site and method of administration, scheduling of administration, and other factors known to medical practitioners. In human therapy, it is important to provide a dosage form that delivers the required therapeutic amount of the each therapeutic agent in vivo, and renders therapeutic agent bioavailable in a rapid manner. In addition to the dosage forms described herein, the dosage forms described by Phillips et al. in U.S. Pat. Nos. 5,840,737, 6,489,346, 6,699,885 and 6,645,988 are incorporated herein by reference.

The percent of intact drug that is absorbed into the bloodstream is not narrowly critical, as long as a therapeutically effective amount, e.g., a gastrointestinal-disorder-effective amount of a proton pump inhibiting agent, is absorbed following administration of the pharmaceutical composition to a subject. Gastrointestinal-disorder-effective amounts in tablets may be found in U.S. Pat. No. 5,622,719. It is understood that the amount of proton pump inhibiting agent and/or buffering agent that is administered to a subject is dependent on a number of factors, e.g., the sex, general health, diet, and/or body weight of the subject.

Illustratively, administration of a substituted bicyclic aryl-imidazole to a young child or a small animal, such as a dog, a relatively low amount of the proton pump inhibitor, e.g., about 1 mg to about 30 mg, will often provide blood serum concentrations consistent with therapeutic effectiveness. Where the subject is an adult human or a large animal, such as a horse, achievement of a therapeutically effective blood serum concentration will require larger dosage units, e.g., about 10 mg, about 15 mg, about 20 mg, about 30 mg, about 40 mg, about 80 mg, or about 120 mg dose for an adult human, or about 150 mg, or about 200 mg, or about 400 mg, or about 800 mg, or about 1000 mg dose, or about 1500 mg dose, or about 2000 mg dose, or about 2500 mg dose, or about 3000 mg dose or about 3200 mg dose or about 3500 mg dose for an adult horse.

In various other embodiments of the present invention, the amount of proton pump inhibitor administered to a subject is, e.g., about 0.5-2 mg/Kg of body weight, or about 0.5 mg/Kg of body weight, or about 1 mg/Kg of body weight, or about 1.5 mg/Kg of body weight, or about 2 mg/Kg of body weight.

Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route chosen for administration, the condition of the particular subject.

In various embodiments, unit dosage forms for humans contain about 1 mg to about 120 mg, or about 1 mg, or about 5 mg, or about 10 mg, or about 15 mg, or about 20 mg, or about 30 mg, or about 40 mg, or about 50 mg, or about 60 mg, or about 70 mg, or about 80 mg, or about 90 mg, or about 100 mg, or about 110 mg, or about 120 mg of a proton pump inhibitor.

In a further embodiment of the present invention, the pharmaceutical composition is administered in an amount to achieve a measurable serum concentration of a non-acid degraded proton pump inhibiting agent greater than about 0.1 μg/ml within about 30 minutes after administration of the pharmaceutical composition. In another embodiment of the present invention, the pharmaceutical composition is administered to the subject in an amount to achieve a measurable serum concentration of a non-acid degraded or non-acid reacted proton pump inhibiting agent greater than about 100 ng/ml within about 15 minutes after administration of the pharmaceutical composition. In yet another embodiment, the pharmaceutical composition is administered to the subject in an amount to achieve a measurable serum concentration of a non-acid degraded or non-acid reacted proton pump inhibiting agent greater than about 100 ng/ml within about 10 minutes after administration of the pharmaceutical composition.

In another embodiment of the present invention, the composition is administered to the subject in an amount to achieve a measurable serum concentration of the proton pump inhibiting agent greater than about 150 ng/ml within about 15 minutes and to maintain a serum concentration of the proton pump inhibiting agent of greater than about 150 ng/ml from about 15 minutes to about 1 hour after administration of the composition. In yet another embodiment of the present invention, the composition is administered to the subject in an amount to achieve a measurable serum concentration of the proton pump inhibiting agent greater than about 250 ng/ml within about 15 minutes and to maintain a serum concentration of the proton pump inhibiting agent of greater than about 250 ng/ml from about 15 minutes to about 1 hour after administration of the composition. In another embodiment of the present invention, the composition is administered to the subject in an amount to achieve a measurable serum concentration of the proton pump inhibiting agent greater than about 350 ng/ml within about 15 minutes and to maintain a serum concentration of the proton pump inhibiting agent of greater than about 350 ng/ml from about 15 minutes to about 1 hour after administration of the composition. In another embodiment of the present invention, the composition is administered to the subject in an amount to achieve a measurable serum concentration of the proton pump inhibiting agent greater than about 450 ng/ml within about 15 minutes and to maintain a serum concentration of the proton pump inhibiting agent of greater than about 450 ng/ml from about 15 minutes to about 1 hour after administration of the composition.

In another embodiment of the present invention, the composition is administered to the subject in an amount to achieve a measurable serum concentration of the proton pump inhibiting agent greater than about 150 ng/ml within about 30 minutes and to maintain a serum concentration of the proton pump inhibiting agent of greater than about 150 ng/ml from about 30 minutes to about 1 hour after administration of the composition. In yet another embodiment of the present invention, the composition is administered to the subject in an amount to achieve a measurable serum concentration of the proton pump inhibiting agent greater than about 250 ng/ml within about 30 minutes and to maintain a serum concentration of the proton pump inhibiting agent of greater than about 250 ng/ml from about 30 minutes to about 1 hour after administration of the composition. In another embodiment of the present invention, the composition is administered to the subject in an amount to achieve a measurable serum concentration of the proton pump inhibiting agent greater than about 350 ng/ml within about 30 minutes and to maintain a serum concentration of the proton pump inhibiting agent of greater than about 350 ng/ml from about 30 minutes to about 1 hour after administration of the composition. In another embodiment of the present invention, the composition is administered to the subject in an amount to achieve a measurable serum concentration of the proton pump inhibiting agent greater than about 450 ng/ml within about 30 minutes and to maintain a serum concentration of the proton pump inhibiting agent of greater than about 450 ng/ml from about 30 minutes to about 1 hour after administration of the composition.

In still another embodiment of the present invention, the composition is administered to the subject in an amount to achieve a measurable serum concentration of a non-acid degraded or non-acid reacted proton pump inhibiting agent greater than about 500 ng/ml within about 1 hour after administration of the composition. In yet another embodiment of the present invention, the composition is administered to the subject in an amount to achieve a measurable serum concentration of a non-acid degraded or non-acid reacted proton pump inhibiting agent greater than about 300 ng/ml within about 45 minutes after administration of the composition.

Contemplated compositions of the present invention provide a therapeutic effect as proton pump inhibiting agent medications over an interval of about 5 minutes to about 24 hours after administration, enabling, for example, once-a-day, twice-a-day, three times a day, etc. administration if desired.

Generally speaking, one will desire to administer an amount of the compound that is effective to achieve a serum level commensurate with the concentrations found to be effective in vivo for a period of time effective to elicit a therapeutic effect. Determination of these parameters is well within the skill of the art. These considerations are well known in the art and are described in standard textbooks.

In one embodiment of the present invention, the composition is administered to a subject in a gastrointestinal-disorder-effective amount, that is, the composition is administered in an amount that achieves a therapeutically-effective dose of a proton pump inhibiting agent in the blood serum of a subject for a period of time to elicit a desired therapeutic effect. Illustratively, in a fasting adult human (fasting for generally at least 10 hours) the composition is administered to achieve a therapeutically-effective dose of a proton pump inhibiting agent in the blood serum of a subject within about 45 minutes after administration of the composition. In another embodiment of the present invention, a therapeutically-effective dose of the proton pump inhibiting agent is achieved in the blood serum of a subject within about 30 minutes from the time of administration of the composition to the subject. In yet another embodiment, a therapeutically-effective dose of the proton pump inhibiting agent is achieved in the blood serum of a subject within about 20 minutes from the time of administration to the subject. In still another embodiment of the present invention, a therapeutically-effective dose of the proton pump inhibiting agent is achieved in the blood serum of a subject at about 15 minutes from the time of administration of the composition to the subject.

In further embodiments, greater than about 98%; or greater than about 95%; or greater than about 90%; or greater than about 75%; or greater than about 50% of the drug absorbed into the bloodstream is in a non-acid degraded or a non-acid reacted form.

In other embodiments, the pharmaceutical compositions provide a release profile of the proton pump inhibitor, using USP dissolution methods, whereby greater than about 50% of the proton pump inhibitor is released from the composition within about 2 hours; or greater than 50% of the proton pump inhibitor is released from the composition within about 1.5 hours; or greater than 50% of the proton pump inhibitor is released from the composition within about 1 hour after exposure to gastrointestinal fluid. In another embodiment, greater than about 60% of the proton pump inhibitor is released from the composition within about 2 hours; or greater than 60% of the proton pump inhibitor is released from the composition within about 1.5 hours; or greater than 60% of the proton pump inhibitor is released from the composition within about 1 hour after exposure to gastrointestinal fluid. In yet another embodiment, greater than about 70% of the proton pump inhibitor is released from the composition within about 2 hours; or greater than 70% of the proton pump inhibitor is released from the composition within about 1.5 hours; or greater than 70% of the proton pump inhibitor is released from the composition within about 1 hour after exposure to gastrointestinal fluid.

Prokinetic Agents

The prokientic agent is administered and dosed in accordance with good medical practice, taking into account the clinical condition of the individual patient, the site and method of administration, scheduling of administration, and other factors known to medical practitioners. According to one aspect of the invention, the pharmaceutical composition comprises two different prokinetic agents. According to another aspect of the invention, the pharmaceutical compositon comprises two different prokinetic agents wherein at least one of the prokinetic agents is a 5HT inhibitor.

In human therapy, it is important to provide a dosage form that delivers the required therapeutic amount of the drug in vivo, and renders the drug bioavailable at the appropriate time. According to one aspect of the invention, part of the prokinetic agent is in an immediate release form and part of the prokinetic agent is in a delayed release form. According to another aspect of the invention, two therapeutically effective doses are present in the pharmaceutical composition, one in an immediate release form and another in a delayed release form. The dosing of prokinetic agents will vary but can be readily determined by one of skill in the art.

Combination Therapies

The compositions and methods described herein may also be used in conjunction with other well known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician. The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

Dosage Form

The pharmaceutical compositions of the present invention contain desired amounts of proton pump inhibitor, a buffering agent and a prokinetic agent and can be in the form of: a tablet, (including a suspension tablet, a chewable tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC) a lozenge, a sachet, a troche, pellets, granules, or an aerosol. These pharmaceutical compositions of the present invention can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, tableting, extruding, coacervation and the like.

In one embodiment, the proton pump inhibitor and prokinetic agent are microencapsulated prior to being formulated into one of the above forms. In another embodiment, all or some of the proton pump inhibitor is microencapsualted prior to being formulated into one of the above forms. In another embodiment, some or all of the buffering agent is microencapsulated prior to being formulated into one of the above forms. In other embodiments, all or some of the prokinetic agent is microencapsulated prior to being further formulated into one of the above forms. In still another embodiment, some or all of the prokinetic agent is coated prior to being further formulated into one of the above forms by using standard coating procedures, such as those described in Remington's Pharmaceutical Sciences, 20th Edition (2000). In yet other embodiments contemplated by the present invention, a film coating is provided around the pharmaceutical composition.

In other embodiments, the pharmaceutical compositions further comprise one or more additional materials such as a pharmaceutically compatible carrier, binder, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

Parietal cell activators are administered in an amount sufficient to produce the desired stimulatory effect without causing untoward side effects to patients. In one embodiment, the parietal cell activator is administered in an amount of about 5 mg to about 2.5 grams per 20 mg dose of the proton pump inhibitor.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, e.g., diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Exemplary Solid Oral Dosage Compositions

Solid oral dosage compositions, e.g., tablets, chewable tablets, effervescent tablets, caplets, and capsules, are prepared by mixing the proton pump inhibitor, one or more buffering agent, at least one prokinetic agent, and pharmaceutical excipients to form a bulk blend composition. When referring to these bulk blend compositions as homogeneous, it is meant that the proton pump inhibitor, buffering agent, and prokinetic agent are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent.

Compressed tablets are solid dosage forms prepared by compacting the bulk blend compositions described above. In various embodiments, compressed tablets of the present invention will comprise one or more functional excipients such as binding agents and/or disintegrants. In other embodiments, the compressed tablets will comprise a film surrounding the final compressed tablet. In other embodiments, the compressed tablets comprise one or more excipients and/or flavoring agents.

A chewable tablet may be prepared by compacting bulk blend compositions, described above. In one embodiment, the chewable tablet comprises a material useful for enhancing the shelf-life of the pharmaceutical composition. In another embodiment, microencapsulated material has taste-masking properties. In various other embodiments, the chewable tablet comprises one or more flavoring agents and one or more taste-masking materials. In yet other embodiments the chewable tablet comprised both a material useful for enhancing the shelf-life of the pharmaceutical formulation and one or more flavoring agents.

In various embodiments, the microencapsulated proton pump inhibitor, buffering agent, prokinetic agent, and optionally one or more excipients, are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the buffering agent and the proton pump inhibitor into the gastrointestinal fluid. When at least 50% of the pharmaceutical composition has disintegrated, the compressed mass has substantially disintegrated.

A capsule may be prepared by placing the bulk blend composition, described above, inside a capsule.

Exemplary Powder Compositions

A powder for suspension may be prepared by combining proton pump inhibitor, one or more buffering agent and one or more prokinetic agents. In various embodiments, the powder may comprise one or more pharmaceutical excipients and flavors. Powder for suspension is prepared by mixing the proton pump inhibitor, one or emore buffering agetns, one or more prokinetic agents, and optional pharmaceutical excipients to form a bulk blend composition. This bulk blend is uniformly subdivided into unit dosage packaging or multi-dosage packaging units. The term “uniform” means the homogeneity of the bulk blend is substantially maintained during the packaging process.

In some embodiments, some or all of the proton pump inhibitor is micronized. In other embodiments, some or all of the prokinetic agent is micronized. Additional embodiments of the present invention also comprise a suspending agent and/or a wetting agent.

Effervescent powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to known technology for tablet preparation.

Wet granulation is one the oldest methods of granule preparation. The individual steps in the wet granulation process of tablet preparation include milling and sieving of the ingredients, dry powder mixing, wet massing, granulation, and final grinding. In various embodiments, the microencapsulated PPI is added to the other excipients of the pharmaceutical composition after they have been wet granulated.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the microencapsulated PPI is dry granulated with other excipients in the pharmaceutical composition. In other embodiments, the microencapsulated omeprazole is added to other excipients of the pharmaceutical composition after they have been dry granulated.

Powder for Suspension

Compositions are provided comprising a pharmaceutical composition comprising at least one proton pump inhibitor, at least one buffering agent, at least one prokinetic agent, and at least one suspending agent for oral administration to a subject. The composition may be a powder for suspension, and upon admixture with water, a substantially uniform suspension is obtained.

A suspension is “substantially uniform” when it is mostly homogenous, that is, when the suspension is composed of approximately the same concentration of proton pump inhibitor at any point throughout the suspension. A suspension is determined to be composed of approximately the same concentration of proton pump inhibitor throughout the suspension when there is less than about 20%, less than about 15%, less than about 13%, less than about 11%, less than about 10%, less than about 8%, less than about 5%, or less than about 3% variation in concentration among samples taken from various points in the suspension.

The concentration at various points throughout the suspension can be determined by any suitable means known in the art. For example, one suitable method of determining concentration at various points involves dividing the suspension into three substantially equal sections: top, middle and bottom. The layers are divided starting at the top of the suspension and ending at the bottom of the suspension. Any number of sections suitable for determining the uniformity of the suspension can be used, such as for example, two sections, three sections, four sections, five sections, or six or more sections. The sections can be named in any appropriate manner, such as relating to their location (e.g., top, middle, bottom), numbered (e.g., one, two, three, four, five, six, etc.), or lettered (e.g., A, B, C, D, E, F, G, etc.). The sections can be divided in any suitable configuration. In one embodiment, the sections are divided from top to bottom, which allows a comparison of sections from the top and sections from the bottom in order to determine whether and at what rate the proton pump inhibitor is settling into the bottom sections. Any number of the assigned sections suitable for determining uniformity of the suspension can be evaluated, such as, e.g., all sections, 90% of the sections, 75% of the sections, 50% of the sections, or any other suitable number of sections.

Concentration is easily determined by methods known in the art, such as, e.g., methods described herein. In one embodiment, concentration is determined using percent label claim. “Percent label claim” (% label claim) is calculated using the actual amount of proton pump inhibitor or prokinetic agent per sample compared with the intended amount of proton pump inhibitor or prokinetic agent per sample. The intended amount of proton pump inhibitor or prokinetic agent per sample can be determined based on the formulation protocol or from any other suitable method, such as, for example, by referencing the “label claim,” that is, the intended amount of proton pump inhibitor or prokinetic agent depicted on labeling complying with the regulations promulgated by the United States Food and Drug Administration.

In one aspect of the present invention, the suspension is divided into sections and the percent label claim is determined for each section. The suspension is determined to be substantially uniform if the suspension comprises at least one of (a) at least about a set threshold percent label claim throughout the evaluated sections or (b) has less than a set percentage variation in percent label claim throughout the evaluated sections. The suspension can comprise either (a) or (b) or can comprise both (a) and (b). The evaluated sections of the suspension can have any set threshold percent label claim suitable for determining that the suspension is substantially uniform. For example, the sections can comprise, e.g., at least about 70, at least about 75, at least about 80, at least about 85, at least about 87, at least about 88, at least about 89, at least about 90, at least about 93, at least about 95, at least about 98, at least about 100, at least about 105, at least about 110, at least about 115 percent label claim of proton pump inhibitor or any range that falls therein, such as, e.g., from about 80 to about 115, from about 85 to about 110, from about 87 to about 108, from about 89 to about 106, from about 90 to about 105, and so on, percent label claim of proton pump inhibitor. The evaluated sections of the suspension can have less than any set percentage variation in percent label claim suitable for determining that the suspension is substantially uniform, such as, e.g., about 25%, about 20%, about 17%, about 15%, about 13%, about 11%, about 10%, about 7%, about 5%, about 3% or about 0% variation.

In another aspect of the present invention, the suspension is substantially uniform if it comprises at least one of (a) at least about 87% label claim of proton pump inhibitor in top, middle and bottom sections determined by separating the suspension into three substantially equal sections from top to bottom for at least about five minutes after admixture with water, or (b) less than about 11% variation in % label claim among each of the top, middle and bottom sections for at least about five minutes after admixture with water.

In an alternate aspect of the present invention, the suspension is substantially uniform if it comprises at least one of (a) at least about 80% label claim of proton pump inhibitor in top, middle and bottom sections determined by separating the suspension into three substantially equal sections from top to bottom for at least about 60 minutes after admixture with water, or (b) less than about 15% variation in % label claim among each of the top, middle and bottom sections for at least about sixty minutes after admixture with water.

In some embodiments, the composition will remain substantially uniform for a suitable amount of time corresponding to the intended use of the composition, such as, e.g., for at least about 5 minutes, about 10 minutes, about 15 minutes, about 20 minutes, about 30 minutes, about 45 minutes, about 60 minutes (I hour), about 75 minutes, about 90 minutes, about 105 minutes, about 120 minutes (2 hours), about 150 minutes, about 180 minutes (3 hours), about 210 minutes, about 4 hours, about 5 hours or more after admixture with water. In one embodiment, the suspension remains substantially uniform from about 5 minutes to about 4 hours after admixture with water. In another embodiment, the suspension remains substantially uniform from about 15 minutes to about 3 hours after admixture with water. In yet another embodiment, the suspension is remains substantially uniform from at least about 1 to at least about 3 hours after admixture with water.

In one embodiment of the present invention, the composition will remain substantially uniform at least until the suspension is prepared for administration to the patient. The suspension can be prepared for administration to the patient at any time after admixture as long as the suspension remains substantially uniform. In another embodiment, the suspension is prepared for administration to the patient from any time after admixture until the suspension is no longer uniform. For example, the suspension can be prepared for administration to the patient from about 5 minutes, about 10 minutes, about 15 minutes, about 20 minutes, about 30 minutes, about 45 minutes, about 60 minutes (1 hour), about 75 minutes, about 90 minutes, about 105 minutes, about 120 minutes (2 hours), about 150 minutes, about 180 minutes (3 hours), about 210 minutes, about 4 hours, about 5 hours or more after admixture with water. In one embodiment, the suspension is prepared for administration to the patient from about 5 minutes to about 4 hours after admixture. In another embodiment, the suspension is prepared for administration to the patient from about 15 minutes to about 3 hours after admixture. In yet another embodiment, the suspension is prepared for administration to the patient from at least about 1 to at least about 3 hours after admixture.

In an alternate embodiment, the composition remains substantially uniform until the composition is actually administered to the patient. The suspension can be administered to the patient at any time after admixture as long as the suspension remains substantially uniform. In one embodiment, the suspension is administered to the patient from any time after admixture until the suspension is no longer uniform. For example, the suspension can be administered to the patient from about 5 minutes, about 10 minutes, about 15 minutes, about 20 minutes, about 30 minutes, about 45 minutes, about 60 minutes (1 hour), about 75 minutes, about 90 minutes, about 105 minutes, about 120 minutes (2 hours), about 150 minutes, about 180 minutes (3 hours), about 210 minutes, about 4 hours, about 5 hours or more after admixture with water. In one embodiment, the suspension is administered to the patient from about 5 minutes to about 4 hours after admixture. In another embodiment, the suspension is administered to the patient from about 15 minutes to about 3 hours after admixture. In yet another embodiment, the suspension is administered to the patient from at least about 1 to at least about 3 hours after admixture.

In one embodiment, the composition comprises at least one proton pump inhibitor, at least one buffering agent, at least one prokinetic agent, and xanthan gum. The composition is a powder for suspension, and upon admixture with water, a first suspension is obtained that is substantially more uniform when compared to a second suspension comprising the proton pump inhibitor, the buffering agent, the prokinetic agent, and suspending agent, wherein the suspending agent is not xanthan gum. In one embodiment, the first suspension comprises at least one of (a) at least about 87% label claim of proton pump inhibitor in top, middle and bottom sections determined by separating the suspension into three substantially equal sections from top to bottom for at least about five minutes after admixture with water, or (b) less than about 11% variation in % label claim among each of the top, middle and bottom sections for at least about five minutes after admixture with water.

In another embodiment, the first suspension comprises at least one of (a) at least about 80% label claim of proton pump inhibitor in top, middle and bottom sections determined by separating the suspension into three substantially equal sections from top to bottom for at least about sixty minutes after admixture with water, or (b) less than about 15% variation in % label claim among each of the top, middle and bottom sections for at least about sixty minutes after admixture with water.

In one embodiment, the composition comprises omeprazole, sodium bicarbonate and xanthan gum. The composition is a powder for suspension, and upon admixture with water, a substantially uniform suspension is obtained. In one embodiment, the suspension comprises at least one of (a) at least about 87% label claim of proton pump inhibitor in top, middle and bottom sections determined by separating the suspension into three substantially equal sections from top to bottom for at least about five minutes after admixture with water, or (b) less than about 11% variation in % label claim among each of the top, middle and bottom sections for at least about five minutes after admixture with water. In another embodiment, the suspension comprises at least one of (a) at least about 80% label claim of proton pump inhibitor in top, middle and bottom sections determined by separating the suspension into three substantially equal sections from top to bottom for at least about sixty minutes after admixture with water, or (b) less than about 15% variation in % label claim among each of the top, middle and bottom sections for at least about sixty minutes after admixture with water.

In yet another embodiment, the composition comprises omeprazole, sodium bicarbonate, at least one prokinetic agent, xanthan gum, and at least one sweetener or flavoring agent. The composition is a powder for suspension. Upon admixture with water, a substantially uniform suspension is obtained. In one embodiment, the suspension comprises at least one of (a) at least about 87% label claim of proton pump inhibitor in top, middle and bottom sections determined by separating the suspension into three substantially equal sections from top to bottom for at least about five minutes after admixture with water, or (b) less than about 11% variation in % label claim among each of the top, middle and bottom sections for at least about five minutes after admixture with water. In another embodiment, the suspension comprises at least one of (a) at least about 80% label claim of proton pump inhibitor in top, middle and bottom sections determined by separating the suspension into three substantially equal sections from top to bottom for at least about sixty minutes after admixture with water, or (b) less than about 15% variation in % label claim among each of the top, middle and bottom sections for at least about sixty minutes after admixture with water.

Other Exemplary Compositions

Pharmaceutical compositions suitable for buccal or sublingual administration include intra-oral batch or solid dosage forms, e.g., lozenges. Other types of release delivery systems are available and known to those of skill in the art. Examples of such delivery systems include, but are not limitd to: polymer-based systems such as polylactic acid, polyglycolic acid, polyanhydrides and polycaprolactone; nonpolymer-based systems that are lipids, including sterols such as cholesterol, cholesterol esters and fatty acids, or neutral fats, such as mono-, di- and triglycerides; hydrogel release systems; silastic systems; peptide-based systems; wax coatings; compressed tablets using conventional binders and excipients partially fused implants and the like. See, e.g., Liberman et al., Pharmaceutical Dosage Forms, 2 Ed., Vol. 1, pp. 209-214 (1990).

For the sake of brevity, all patents and other references cited herein are incorporated by reference in their entirety.

### EXAMPLES

The present invention is further illustrated by the following examples, which should not be construed as limiting in any way. The experimental procedures to generate the data shown are discussed in more detail below. For all formulations herein, multiple doses may be proportionally compounded as is known in the art. The coatings, layers, and encapsulations are applied in conventional ways using equipment customary for these purposes.

The invention has been described in an illustrative manner, and it is to be understood that the terminology used is intended to be in the nature of description rather than of limitation.

### Example 1

### Spinning Disk Microencapsulation Process

The basic operation for the spinning disk-solvent process used is as follows: An encapsulation solution is prepared by dissolving the encapsulation material in the appropriate solvent. Proton pump inhibitor (PPI) in combination with buffering agent and prokinetic agent, or proton pump inhibitor alone if intended to be microencapsulated and then combined with a buffering agent and prokinetic agent, is dispersed in the coating solution and fed onto the center of the spinning disk. A thin film is produced across the surface of the disk and atomization occurs as the coating material left the periphery of the disk. The microspheres are formed by removal of the solvent using heated airflow inside the atomization chamber and collected as a free-flowing powder using a cyclone separator.

### Example 2

### Spray Drying Microencapsulation Process

A spray dryer consists of the same components as a spinning disk except atomization is achieved through an air nozzle instead of a spinning disk.

### Example 3

### Preparation of Powder for Suspension for Oral Dosing

Powder for suspension (liquid oral pharmaceutical composition) according to the present invention, is prepared by mixing PPI (40 mg omeprazole in the form of microencapsulated omeprazole, omeprazole powder or omeprazole base) with at least one buffering agent and a prokinetic agent. In one embodiment, omeprazole or other proton pump inhibitor, which can be obtained from powder, capsules, tablets, or from the solution for parenteral administration, is mixed with sodium bicarbonate (1680 mg), prokinetic agent, and sweeteners and flavors.

### Example 4

### Microencapsulated Proton Pump Inhibitor

The amount of microencapsulated omeprazole used in each tablet batch varies based on the actual payload of each set of microcapsules to achieve the theoretical dose of 40 mg. The omeprazole is microencapsulated in a similar manner as that described in Example 1 and Example 2. All ingredients are mixed well to achieve a homogenious blend.

Omeprazole microspheres were prepared using a high-speed rotary tablet press (TBCB Pharmaceutical Equipment Group, Model ZPY15). Round, convex tablets with diameters of about 10 mm and an average weight of approximately 600 mg per tablet were prepared.

TABLE 4.A

| No | Microencapsulation Material | Method | Size | |
|---|---|---|---|---|
| 1 | Myverol | Disk-hot melt | 120-200 | micron |
| 2 | Myverol | Disk-hot melt | 120-200 | micron |
| 3 | KLX & BHT (0.1% of KLX) | Disk-hot melt | 25-125 | micron |
| 4 | KLX & BHT (0.1% of KLX) | Disk-hot melt | 25-125 | micron |
| 5 | Methocel A15LV & PEG 3350 (5%) | Spray dry | 5-30 | micron |
| 6 | Methocel A15LV, PEG 300 (5%) & BHT (0.1%) | Spray dry | 5-30 | micron |
| 7 | Methocel A15LV, Span 20 (5%) & BHT (0.1%) | Spray dry | 5-30 | micron |
| 8 | Methocel A15LV BHT (0.1%) | Spray dry | 5-30 | micron |
| 9 | Modified food starch, PEG 3350 (2.5%) & BHT (0.1%) | Spray dry | 5-30 | micron |
| 10 | Methocel A15LV, PEG 3350 (5%), BHT (0.1%) & Sodium | Spray dry | 5-30 | micron |
| | bicarbonate | | | |
| 11 | Opadry YS-1-7003 PEG 3350 (5%) BHT (0.1%) | Spray dry | 5-30 | micron |
| 12 | Methocel K4M PEG 3350 (10%) BHT | Spray dry | 5-30 | micron |
| 13 | Kollicoat IR, PEG 3350 (5%) & BHT | Spray dry | 5-30 | micron |
| 14 | Eudragit RD 100, PEG 3350 (5%) & BHT (0.1%) | Spray dry | 5-30 | micron |
| 15 | Klucel (HPC), PEG 3350 (5%) & BHT (0.1%) | Spray dry | 5-30 | micron |
| 16 | Ethocel | Disk-solvent | 25-125 | micron |
| 17 | Ethocel (50%) Methocel E5 (50%) | Disk-solvent | 25-125 | micron |
| 18 | Ethocel (75%) Methocel (25%) | Disk-solvent | 25-125 | micron |
| 19 | Methocel | Disk-solvent | 25-125 | micron |
| 20 | Ethocel Sodium Bicarbonate | Disk-solvent | 25-125 | micron |
| 21 | Ethocel & PEG 3350 (5%) | Disk-solvent | 25-125 | micron |
| 22 | Ethocel (50%) & Klucel EXAF (50%) | Disk-solvent | 25-125 | micron |
| 23 | Klucel | Disk-solvent | 25-100 | micron |
| 24 | Sepifilm LP | Disk-solvent | 25-100 | micron |
| 25 | Eudragit E100 | Disk-solvent | 25-80 | micron |
| 26 | Eudragit E100 | Disk-solvent | 25-80 | micron |
| 27 | Eudragit E100 & Span 20 (5%) | Disk-solvent | 25-80 | micron |
| 28 | Eudragit E100 & PEG 300 (5%) | Disk-solvent | 25-80 | micron |
| 29 | Eudragit EPO | Disk-solvent | 25-80 | micron |
| 30 | Eudragit EPO | Disk-solvent | 25-90 | micron |
| 31 | Opadry AMB | Spray dry | <30 | micron |
| 32 | Sucralose | Spray dry | | |
| 33 | Sepifilm LP | Spray dry | | |
| 34 | Kollicoat IR | Spray dry | | |
| 35 | Kollicoat IR & Sodium bicarbonate | Spray dry | <30 | micron |
| 36 | Klucel & Sucralose (20%) | Spray dry | | |
| 37 | Klucel & Sucrose (20%) | Spray dry | | |
| 38 | Klucel & Sodium bicarbonate | Spray dry | <30 | micron |
| 39 | Klucel(60%) Sucraolse (10%) Sodium bicarbonate (30%) | Spray dry | <50 | micron |
| 40 | Eudragit EPO | Disk-solvent | 20-75 | micron |
| 41 | Eudragit EPO | Disk-solvent | 20-90 | micron |
| 42 | Eudragit EPO(67%) Sodium bicarb(33%) | Disk-solvent | 20-85 | micron |
| 43 | EudragitEPO(61.5%) PEG 300(11.5%) PEG 3350 (3.8%) | Disk-solvent | 20-110 | micron |
| | Sod Bicarb (23.2%) | | | |
| 44 | Eudragit EPO | Disk-solvent | 20-100 | micron |
| 45 | Opadry AMB (No TiO₂) | Spray dry | | |
| 46 | Opadry AMB (No TiO₂) | Spray dry | | |
| 47 | Opadry AMB (No TiO₂) BHT (0.1%) | Spray dry | | |
| 48 | Cavamax W8 (gamma-CD) | Spray dry (pH = 10) | 5-30 | microns |
| 49 | Cavamax W8 & L-lysine | Spray dry (pH = 10) | 5-30 | micron |
| 50 | Cavamax W8 & Methocel A15 LV | Spray dry (pH = 10) | 5-40 | micron |
| 52 | Opadry AMB & BHT | Spray Dry (aqueous) | 5-30 | micron |

Stability studies were performed on the microencapsulated omeprazole. The various tablets used in the stability studies were manufactured using the following materials: Encapsulated omeprazole, sodium bicarbonate (1260 mg), calcium carbonate (790 mg), croscarmellose sodium (64 mg), Klucel (160 mg), Xylitab 100 (380 mg), microcrystalline cellulose (128 mg), sucralose (162 mg), peppermint duraromer (34 mg), peach duraromer (100 mg), masking powder (60 mg), FD&C Lake No. 40 Red (3 mg), and magnesium stearate (32 mg). An exemplary formulation used to make each of the tablets, as well as the blending methods used, are shown in Table 4.B., below.

TABLE 4.B

| | | | | Feed | |
|---|---|---|---|---|---|
| | Method and | | | Rate | Inlet/Outlet |
| Sample | Solvent | Microencapsulation Material | Wt % of material | (g/min) | Temp(° C.) |
| 53 | Spray dry* | Methocel A15 LV | 5% | 4.2 | 125/70 |
| | Water | PEG 3350 | | | |
| 54 | Spray dry | Methocel A15 LV | 5% | 4.0 | 125/70 |
| | Water | BHT | | | |
| 55 | Spray dry | Opadry YS-1-7003 | 5% | 4.2 | 126/60 |
| | Water | PEG 3350 | | | |
| | | BHT | | | |
| 56 | Spray dry | Kollicoat IR | 10% | 3.0 | 128/85 |
| | Water | PEG 3350 | | | |
| | | BHT | | | |
| 57 | Spray dry | Eudragit RD100 | 5% | 4.0 | 127/87 |
| | Water | PEG 3350 | | | |
| | | BHT | | | |
| 58 | Spray dry | Klucel | 5% | 4.2 | 126/83 |
| | Water | PEG 3350 | | | |
| | | BHT | | | |
| 59 | Spinning disk** | Klucel | 10% | 90 | /52 |
| | 75% Methanol | | | | |
| | 25% Acetone | | | | |
| 60 | Spray dry | Kollicoat | 5% | 4.5 | 129/86 |
| | Water | Sodium Bicarb | | | |
| 61 | Spray dry | Klucel | 5% | 4.5 | 122/84 |
| | Water | Sodium Bicarb | | | |
| 62 | Spinning disk | Eudragit EPO | 10% | 90 | /50 |
| | 75% Methanol | | | | |
| | 25% Acetone | | | | |
| 63 | Spray dry | Opadry AMB | 10% | 4.4 | 124/79 |
| | Water | BHT | | | |

*Used a concentric nozzle with 0.055 inch air opening and a 0.028 inch fluid opening.
**Used a 3-inch stainless steel disk rotating at approximately 4,500 rpm.

### Example 5

### Stability of Microencapsulated Omeprazole

The tablets used in the stability study were packaged into 60 ml HDPE 33/400 bottles with two 1 gram, 2 in 1 desiccant canisters. The HDPE bottles were closed hand tight and induction sealed using a 33/400 CRC SFGD 75M cap with a polypropylene liner. Samples were placed in controlled environmental chambers which were maintained at 25≅2° C./60±5% RH and 40±2° C./75±5% RH.

Microspheres that exhibited dissolution results with greater than 80% omeprazole release after 2 hours were placed on stability. The microspheres were stored in opened vials at 25° C. All samples showed degradation after 4 weeks at elevated temperatures. The open vials stored at 25° C. were analyzed after 6-8 weeks for potency and for impurities using the Omeprazole EP method. The stability results are summarized in the Table 5.A.

TABLE 5.A

| | OME Loading | 4-Week Potency Values | AUC |
|---|---|---|---|
| Microencapsulation Material | (Initial) | (Omeprazole Loading) | Purity* |
| Methocel A15LV & PEG 3350 (5%) | 23.3 | 25.0 (107% of initial) @25° C. | 95.65 |
| Methocel A15LV, PEG 300 (5%) & BHT (0.1%) | 26.0 | 24.9( 95.8% of initial) @25° C. | 99.90 |
| Methocel A15LV BHT (0.1%) | 24.8 | 26.4 (106.6% of initial) @25° C. | 99.95 |
| Methocel A15LV, PEG 3350 (5%), BHT (0.1%) & | 2.2 | 2.3 (106% of initial) @25° C. | 76.16 |
| Sodium bicarbonate | | | |
| Opadry YS-1-7003 PEG 3350 (5%) BHT (0.1%) | 20.5 | 22.6 (110% of initial) @25° C. | 100.0 |
| Kollicoat IR, PEG 3350 (5%) & BHT | 26.2 | 23.8 (90.8% of initial) @25° C. | 99.54 |
| Eudragit RD 100, PEG 3350 (5%) & BHT (0.1%) | 21.3 | 19.1 (89.5% of initial) @25° C. | 98.88 |
| Klucel (HPC), PEG 3350 (5%) & BHT (0.1%) | 26.0 | 22.8 (87.8% of initial) @25° C. | 99.70 |
| Ethocel (50%) Methocel E5 (50%) | 25.8 | 21.9 (84.9% of initial) @25° C. | 98.22 |
| | | | (99.3@T₀) |
| Klucel | 22.2 | 20.7 (93.2% of initial) @25° C. | 97.69 |
| Kollicoat IR & Sodium bicarbonate | 26.0 | 21.7 (83.6% of initial) @25° C. | 97.88 |

*AUC Purity = Area Under the Curve after 6-8 weeks at 25° C. in open container.

### Example 6

### Capsule Formulations

The following specific formulations are provided by way of reference only and are not intended to limit the scope of the invention. Each formulation contains therapeutically effective doses of PPI and prokinetic agent as well as sufficient buffering agent to prevent acid degradation of at least some of the PPI by raising the pH of gastric fluid. Amounts of buffer are expressed in weight as well as in molar equivalents (mEq). Amounts of prokinetic agents are typically expressed in a per unit dose amount. The capsules are prepared by blending the PPI and prokinetic agent with buffering agents, and homogeneously blending with excipients as shown in Tables 6.A. to 6.H. below. The appropriate weight of bulk blend composition is filled into a hard gelatine capsule (e.g., size 00) using an automatic encapsualtor (H & K 1500 or MG2 G60).

TABLE 6.A
Omeprazole (20 mg)-Metoclopramide (10 mg) Capsule

| PPI | Buffering Agent | Prokinetic Agent | Excipient |
|---|---|---|---|
| 20 mg | 17.1 mEq or 500 mg | 10 mg | 50 mg Ac-Di-Sol |
| omeprazole | Mg(OH)₂ | Metoclopramide | 30 mg Klucel |
| per capsule | 3.0 mEq or 250 | per capsule | 10 mg |
| | mg NaHCO₃ | | magnesium |
| | 20.1 mEq or 750 mg | | stearate |
| | total buffer | | |

TABLE 6.B
Ompeprazole (40 mg)-Cisapride (10 mg) Capsule

| PPI | Buffering Agent | Prokinetic Agent | Excipient |
|---|---|---|---|
| 40 mg | 20.6 mEq or 600 mg | 10 mg Cisapride | 40 mg Ac-Di-Sol |
| omeprazole | Mg(OH)₂ | per capsule | 35 mg Klucel |
| per capsule | 4.2 mEq or 350 mg | | 10 mg |
| | NaHCO₃ | | magnesium |
| | 24.8 mEq or 950 | | stearate |
| | mg total buffer | | |

TABLE 6.C
Lansoprazole (15 mg)-Mosapride (5 mg) Capsule

| | | Prokinetic | |
|---|---|---|---|
| PPI | Buffering Agent | Agent | Excipient |
| 15 mg | 17.1 mEq or 500 mg | 5 mg | 30 mg Ac-Di-Sol |
| microencapsulated | Mg(OH)₂ | Mosapride | 15 mg Klucel |
| lansoprazole per | 3.0 mEq or 250 mg | per | 7 mg |
| capsule | NaHCO₃ | capsule | magnesium |
| | 20.7 mEq or 750 mg | | stearate |
| | total buffer | | |

TABLE 6.D
Lansoprazole (30 mg)-Mosapride (2.5 mg) Capsule

| PPI | Buffering Agent | Prokinetic Agent | Excipient |
|---|---|---|---|
| 30 mg | 17.1 mEq or 500 mg | 2.5 mg Mosapride | 20 mg Ac-Di-Sol |
| lansoprazole | Mg(OH)₂ | per capsule | 30 mg Klucel |
| per capsule | 4.2 mEq or 350 mg | | 10 mg |
| | NaHCO₃ | | magnesium |
| | 21.3 mEq or 850 | | stearate |
| | mg total buffer | | |

TABLE 6.E
Omeprazole (60 mg)-Domperidone (10 mgs) Capsule

| PPI | Buffering Agent | Prokinetic Agent | Excipient |
|---|---|---|---|
| 60 mg | 17.1 mEq or 500 mg | 10 mg | 20 mg Ac-Di-Sol |
| ompeprazole | Mg(OH)₂ | Domperidone | 25 mg Klucel |
| per capsule | 3.0 mEq or 250 mg | per capsule | 10 mg |
| | NaHCO₃ | | magnesium |
| | 20.1 mEq or 750 mg | | stearate |
| | total buffer | | |

TABLE 6.F
Omeprazole (60 mg)-Clebopride (10 mg) Capsule

| PPI | Buffering Agent | Prokinetic Agent | Excipient |
|---|---|---|---|
| 60 mg | 17.1 mEq or 500 mg | 10 mg Clebopride | 30 mg Ac-Di-Sol |
| ompeprazole | Mg(OH)₂ | per capsule | 15 mg Klucel |
| per capsule | 3.0 mEq or 250 mg | | 7 mg |
| | NaHCO₃ | | magnesium |
| | 20.1 mEq or 750 mg | | stearate |
| | total buffer | | |

TABLE 6.G
Omeprazole (10 mg)-Clebopride (20 mg) Capsule

| PPI | Buffering Agent | Prokinetic Agent | Excipient |
|---|---|---|---|
| 10 mg | 17.1 mEq or 500 mg | 20 mg Clebopride | 30 mg Ac-Di-Sol |
| ompeprazole | Mg(OH)₂ | per capsule | 15 mg Klucel |
| per capsule | 3.0 mEq or 250 mg | | 7 mg |
| | NaHCO₃ | | magnesium |
| | 20.1 mEq or 750 mg | | stearate |
| | total buffer | | |

TABLE 6.H
Omeprazole (40 mg)-Enteric Coated Norcisapride (10 mg) Capsule

| | | Prokinetic | |
|---|---|---|---|
| PPI | Buffering Agent | Agent | Excipient |
| 40 mg | 15.4 mEq or 450 | 10 mg | 30 mg Ac-Di-Sol |
| microencapsulated | mg Mg(OH)₂ | Norcisapride | 7 mg |
| ompeprazole per | 2.4 mEq or 200 | per capsule | magnesium |
| capsule | mg NaHCO₃ | | stearate |
| | 17.8 mEq or 650 | | |
| | mg total buffer | | |

### Example 7

### Tablet Formulations

The following specific formulations are provided by way of reference only and are not intended to limit the scope of the invention. Each formulation contains therapeutically effective doses of PPI and prokinetic agent as well as sufficient buffering agent to prevent acid degradation of at least some of the PPI by raising the pH of gastric fluid. Amounts of buffer are expressed in weight as well as in molar equivalents (mEq). Amounts of prokinetic agents are typically expressed in a per unit dose amount. The tablets are prepared by blending the PPI and prokinetic agent with buffering agents, and homogeneously blending with excipients as shown in Tables 7.A. to 7.H. below. The appropriate weight of bulk blended composition is compressed using l2-inch FFBE toolings in a rotary press (Manesty Epxress) to achieve a hardness of 20-24 kPa.

TABLE 7.A
Ompeprazole (20 mg)-Norcisapride (10 mg) Tablet

| PPI | Buffering Agent | Prokinetic Agent | Excipient |
|---|---|---|---|
| 20 mg | 13.7 mEq or 400 mg | 10 mg | 30 mg Ac-Di-Sol |
| omeprazole | Mg(OH)₂ | Norcisapride | 80 mg Klucel |
| per tablet | 3.0 mEq or 250 mg | per tablet | 10 mg |
| | NaHCO₃ | | magnesium |
| | 16.7 mEq or 650 | | stearate |
| | mg total buffer | | |

TABLE 7.B
Omeprazole (40 mg)-Clebopride (20 mg) Tablet

| | | Prokinetic | |
|---|---|---|---|
| PPI | Buffering Agent | Agent | Excipient |
| 40 mg | 17.1 mEq or 500 | 20 mg | 20 mg Ac-Di-Sol |
| microencapsulated | mg Mg(OH)₂ | Clebopride | 80 mg Klucel |
| omeprazole per | 3.0 mEq or 250 | per tablet | 10 mg |
| tablet | mg NaHCO₃ | | magnesium |
| | 20.1 mEq or 850 | | stearate |
| | mg total buffer | | |

TABLE 7.C
Lansoprazole (15 mg)-Clebopride (10 mg) Tablet

| | | Prokinetic | |
|---|---|---|---|
| PPI | Buffering Agent | Agent | Excipient |
| 15 mg | 17.1 mEq or 500 | 10 mg | 20 mg Ac-Di-Sol |
| microencapsulated | mg Mg(OH)₂ | Clebopride | 80 mg Klucel |
| lansoprazole per | 3.0 mEq or 250 | per tablet | 10 mg |
| tablet | mg NaHCO₃ | | magnesium |
| | 20.1 mEq or 750 | | stearate |
| | mg total buffer | | |

TABLE 7.D
Lansoprazole (30 mg)-Domperidone (10 mg) Tablet

| PPI | Buffering Agent | Prokinetic Agent | Excipient |
|---|---|---|---|
| 30 mg | 20.6 mEq or 500 mg | 10 mg | 20 mg Ac-Di-Sol |
| lansoprazole | Mg(OH)₂ | Domperidone | 80 mg Klucel |
| per tablet | 4.2 mEq or 350 mg | per tablet | 10 mg |
| | NaHCO₃ | | magnesium |
| | 24.8 mEq or 850 mg | | stearate |
| | total buffer | | |

TABLE 7.E
Omeprazole (60 mg)-Mosapride (2.5 mg) Tablet

| PPI | Buffering Agent | Prokinetic Agent | Excipient |
|---|---|---|---|
| 60 mg | 20.6 mEq or 600 mg | 2.5 mg Mosapride | 20 mg Ac-Di-Sol |
| omeprazole | Mg(OH)₂ | per tablet | 80 mg Klucel |
| per tablet | 3.0 mEq or 250 mg | | 10 mg |
| | NaHCO₃ | | magnesium |
| | 23.6 mEq or 850 mg | | stearate |
| | total buffer | | |

TABLE 7.F
Omeprazole (60 mg)-Naproxen (5 mg) Tablet

| PPI | Buffering Agent | Prokinetic Agent | Excipient |
|---|---|---|---|
| 60 mg | 17.1 mEq or 500 mg | 5 mg Mosapride | 20 mg Ac-Di-Sol |
| omprazole | Mg(OH)₂ | per tablet | 60 mg Klucel |
| per tablet | 3.0 mEq or 250 mg | | 10 mg magnesium |
| | NaHCO₃ | | stearate |
| | 20.1 mEq or 850 mg | | |
| | total buffer | | |

TABLE 7.G
Ompeprazole (10 mg)-Cisapride (10 mg) Tablet

| | | Prokinetic | |
|---|---|---|---|
| PPI | Buffering Agent | Agent | Excipient |
| 10 mg | 13.7 mEq or 400 mg | 10 mg | 20 mg Ac-Di-Sol |
| microencapsulated | Mg(OH)₂ | Cisapride | 80 mg Klucel |
| omeprazole per | 3.0 mEq or 250 mg | pre tablet | 10 mg |
| tablet | NaHCO₃ | | magnesium |
| | 16.7 mEq or 650 mg | | stearate |
| | total buffer | | |

TABLE 7.H
Ompeprazole (40 mg)-Metoclopramide (10 mg) Tablet

| PPI | Buffering Agent | Prokinetic Agent | Excipient |
|---|---|---|---|
| 40 mg | 20.6 mEq or 600 | 10 mg | 20 mg |
| microencapsulated | mg Mg(OH)₂ | Metoclopramide | Ac-Di-Sol |
| omeprazole per | 3.0 mEq or 250 | per tablet | 80 mg |
| tablet | mg NaHCO₃ | | Klucel |
| | 23.6 mEq or 850 | | 10 mg |
| | mg total buffer | | magnesium |
| | | | stearate |

### Example 8

### Chewable Tablet Formulations

The following specific formulations are provided by way of reference only and are not intended to limit the scope of the invention. Each formulation contains therapeutically effective doses of PPI and prokinetic agent as well as sufficient buffering agent to prevent acid degradation of at least some of the PPI by raising the pH of gastric fluid. Amounts of buffer are expressed in weight as well as in molar equivalents (mEq). Amounts of prokinetic agents are typically expressed in a per unit dose amount. The tablets are prepared by blending the PPI and prokinetic agent with buffering agents, and homogeneously blending with excipients as shown in Tables 8.A to 8.H. below. The appropriate weight of bulk blended composition is compressed using 5/8-inch FFBE toolings in a rotary press (Manesty Epxress) to achieve a hardness of 17-20 kPa.

TABLE 8.A
Ompeprazole (20 mg)-Mosapride (5 mg) Chewable Tablet

| PPI | Buffering Agent | Prokinetic Agent | Excipient |
|---|---|---|---|
| 20 mg | 20.6 mEq or 600 mg | 5 mg Mosapride per | 170 mg Xylitab |
| microencapsulated | Mg(OH)₂ | tablet | 30 mg Ac-Di-Sol |
| omeprazole per | 5.0 mEq or 420 mg | | 100 mg Klucel |
| tablet | NaHCO₃ | | 40 mg Sucralose |
| | 25.6 mEq or 1020 mg | | 25 mg cherry flavor |
| | total buffer | | 15 mg magnesium |
| | | | stearate |
| | | | 3 mg Red #40 Lake |

TABLE 8.B
Omeprazole (40 mg)-Domperidone (10 mg) Chewable Tablet

| PPI | Buffering Agent | Prokinetic Agent | Excipient |
|---|---|---|---|
| 40 mg | 24.0 mEq or 700 mg | 10 mg Domperidone | 170 mg Dipac sugar |
| microencapsulated | Mg(OH)₂ | per tablet | 30 mg Ac-Di-Sol |
| omeprazole per | 7.1 mEq or 600 mg | | 120 mg Klucel |
| tablet | NaHCO₃ | | 27 mg grape flavor |
| | 27.1 mEq or 1300 mg | | 15 mg magnesium |
| | total buffer | | stearate |
| | | | 1 mg Red #40 Lake |
| | | | 1 mg Blue #2 Lake |

TABLE 8.C
Lansoprazole (15 mg)-Cleopride (10 mg) Chewable Tablet

| PPI | Buffering Agent | Prokinetic Agent | Excipient |
|---|---|---|---|
| 15 mg | 17.1 mEq or 500 | 10 mg cleopride | 170 mg Xylitab |
| lansoprazole | mg Mg(OH)₂ | pre tablet | 30 mg Ac-Di-Sol |
| per tablet | 8.0 mEq or 672 | | 120 mg Klucel |
| | mg NaHCO₃ | | 100 mg Asulfame-K |
| | 25.1 mEq or 1172 | | 27 mg grape flavor |
| | mg total buffer | | 15 mg magnesium |
| | | | stearate |
| | | | 1 mg red #40 lake |
| | | | 1 mg blue #2 lake |

TABLE 8.D
Lansoprazole (30 mg)-Clebopride (20 mg) Chewable Tablet

| | | Prokinetic | |
|---|---|---|---|
| PPI | Buffering Agent | Agent | Excipient |
| 30 mg | 24.0 mEq or 700 mg | 20 mg | 170 mg Xylitab |
| micro- | Mg(OH)₂ | Clebopride | 30 mg Ac-Di-Sol |
| encapsulated | 5.0 mEq or 420 mg | per tablet | 100 mg Klucel |
| lansoprazole | NaHCO₃ | | 25 mg cherry flavor |
| per tablet | 29.0 mEq or 1120 | | 15 mg magnesium |
| | mg total buffer | | stearate |
| | | | 3 mg Red #40 Lake |

TABLE 8.E
Omeprazole (60 mg)-Norcisapride (10 mg) Chewable Tablet

| | | Prokinetic | |
|---|---|---|---|
| PPI | Buffering Agent | Agent | Excipient |
| 60 mg | 15 mEq or 750 mg | 10 mg | 170 mg Xylitab |
| micro- | Ca(OH)₂ | Norcisapride | 30 mg Ac-Di-Sol |
| encapsulated | 15 mEq or 1260 mg | per tablet | 100 mg Klucel |
| omeprazole | NaHCO₃ | | 25 mg cherry flavor |
| per tablet | 30 mEq or 2010 mg | | 15 mg magnesium |
| | total buffer | | stearate |
| | | | 3 mg Red #40 Lake |

TABLE 8.F
Omeprazole (60 mg)-Clebopride (10 mg) Chewable Tablet

| PPI | Buffering Agent | Prokinetic Agent | Excipient |
|---|---|---|---|
| 60 mg | 15 mEq or 750 mg | 10 mg | 170 mg Xylitab |
| omprazole | Ca(OH)₂ | Clebopride | 30 mg Ac-Di-Sol |
| per tablet | 10 mEq or 840 mg | per tablet | 100 mg Klucel |
| | NaHCO₃ | | 15 mg mint flavor |
| | 25 mEq or 1590 mg | | 15 mg magnesium |
| | total buffer | | stearate |

TABLE 8.G
Omeprazole (10 mg)-Metoclopramide (10 mg) Chewable Tablet

| PPI | Buffering Agent | Prokinetic Agent | Excipient |
|---|---|---|---|
| 10 mg | 15 mEq or 750 mg | 10 mg | 170 mg Xylitab |
| omprazole | Ca(OH)₂ | Metoclopramide | 30 mg Ac-Di-Sol |
| per tablet | 10 mEq or 840 mg | per tablet | 100 mg Klucel |
| | NaHCO₃ | | 15 mg mint flavor |
| | 25 mEq or 1590 mg | | 15 mg magnesium |
| | total buffer | | stearate |

TABLE 8.H
Omeprazole (40 mg)-Cisapride (10 mg) Chewable Tablet

| | | Prokinetic | |
|---|---|---|---|
| PPI | Buffering Agent | Agent | Excipient |
| 40 mg | 15 mEq or 750 mg | 10 mg | 170 mg Xylitab |
| micro- | Ca(OH)₂ | Cisapride | 30 mg Ac-Di-Sol |
| encapsulated | 10 mEq or 840 mg | per tablet | 100 mg Klucel |
| omprazole | NaHCO₃ | | 15 mg mint flavor |
| per tablet | 25 mEq or 1590 mg | | 15 mg magnesium |
| | total buffer | | stearate |

### Example 9

### Bite-Disintegration Chewable Tablet Formulations

The following specific formulations are provided by way of reference only and are not intended to limit the scope of the invention. Each formulation contains therapeutically effective doses of PPI and prokinetic agent as well as sufficient buffering agent to prevent acid degradation of at least some of the PPI by raising the pH of gastric fluid. Amounts of buffer are expressed in weight as well as in molar equivalents (mEq). Amounts of prokinetic agent are typically expressed in a per unit dose amount. The tablets are prepared by blending the PPI and prokinetic agent with buffering agents, and homogeneously blending with excipients as shown in Tables 9.A to 9.H. below. The appropriate weight of bulk blended composition is compressed using 5/8-inch FFBE toolings in a rotary press (Manesty Epxress) to achieve a hardness of 8-12 kPa.

TABLE 9.A
Ompeprazole (20 mg)-Metoclopramide (10 mg)
Bite-Disintegration Chewable Tablet

| PPI | Buffering Agent | Prokinetic Agent | Excipient |
|---|---|---|---|
| 20 mg | 20.6 mEq or 600 mg | 10 mg | 60 mg sucralose |
| per | Mg(OH)₂ | Metoclopramide | 60 mg Ac-Di-Sol |
| tablet | 5.0 mEq or 420 mg | per tablet | 60 mg pregelatinized |
| | NaHCO₃ | | starch |
| | 25.6 mEq or 1020 | | 30 mg Klucel |
| | mg total buffer | | 25 mg cherry flavor |
| | | | 15 mg magnesium |
| | | | stearate |
| | | | 3 mg Red #40 Lake |

TABLE 9.B
Omeprazole (40 mg)-Cisapride (10 mg)
Bite-Disintegration Chewable Tablet

| | | Prokinetic | |
|---|---|---|---|
| PPI | Buffering Agent | Agent | Excipient |
| 40 mg | 23.7 mEq or 700 mg | 10 mg | 60 mg sucralose |
| micro- | Mg(OH)₂ | Cisapride | 60 mg Ac-Di-Sol |
| encapsulated | 7.2 mEq or 600 mg | per tablet | 60 mg pregelatinized |
| omeprazole | NaHCO₃ | | starch |
| per tablet | 30.9 mEq or 1300 mg | | 30 mg Klucel |
| | total buffer | | 27 mg grape flavor |
| | | | 15 mg magnesium |
| | | | stearate |
| | | | 1 mg Red #40 Lake |
| | | | 1 mg Blue #2 Lake |

TABLE 9.C
Lansoprazole (15 mg)-Mosapride (5 mg)
Bite-Disintegration Chewable Tablet

| | | Prokinetic | |
|---|---|---|---|
| PPI | Buffering Agent | Agent | Excipient |
| 15 mg | 17.1 mEq or 500 mg | 5 mg | 60 mg sucralose |
| lansoprazole | Mg(OH)₂ | Mosapride | 70 mg Ac-Di-Sol |
| per tablet | 7.2 mEq or 600 mg | per tablet | 70 mg pregelatinized |
| | NaHCO₃ | | starch |
| | 24.2 mEq or 1100 mg | | 30 mg Klucel |
| | total buffer | | 27 mg grape flavor |
| | | | 15 mg magnesium |
| | | | stearate |
| | | | 1 mg Red #40 Lake |
| | | | 1 mg Blue #2 lake |

TABLE 9.D
Lansoprazole (30 mg)-Mosapride (2.5 mg)
Bite-Disintegration Chewable Tablet

| | | Prokinetic | |
|---|---|---|---|
| PPI | Buffering Agent | Agent | Excipient |
| 30 mg | 17.1 mEq or 500 mg | 2.5 mg | 60 mg sucralose |
| micro- | Mg(OH)₂ | Mosapride | 60 mg Ac-Di-Sol |
| encapsulated | 5 mEq or 420 mg | per tablet | 70 mg pregelatinized |
| lansoprazole | NaHCO₃ | | starch |
| per tablet | 22.1 mEq or 1020 mg | | 30 mg Klucel |
| | total buffer | | 25 mg cherry flavor |
| | | | 15 mg magnesium |
| | | | stearate |
| | | | 3 mg Red #40 Lake |

TABLE 9.E
Omeprazole (60 mg)-Domperidone (10 mg)
Bite-Disintegration Chewable Tablet

| | | Prokinetic | |
|---|---|---|---|
| PPI | Buffering Agent | Agent | Excipient |
| 60 mg | 15 mEq or 750 mg | 10 mg | 60 mg sucralose |
| micro- | Ca(OH)₂ | Domperidone | 60 mg Ac-Di-Sol |
| encapsulated | 15 mEq or 1260 mg | per tablet | 60 mg |
| omeprazole | NaHCO₃ | | pregelatinized |
| per tablet | 30 mEq or 2010 mg | | starch |
| | total buffer | | 30 mg Klucel |
| | | | 25 mg cherry flavor |
| | | | 15 mg magnesium |
| | | | stearate |
| | | | 3 mg Red #40 Lake |

TABLE 9.F
Omeprazole (60 mg)-Clebopride (10 mg)
Bite-Disintegration Chewable Tablet

| | | Prokinetic | |
|---|---|---|---|
| PPI | Buffering Agent | Agent | Excipient |
| 60 mg | 15 mEq or 750 mg | 10 mg | 60 mg sucralose |
| omprazole | Ca(OH)₂ | Clebopride | 60 mg Ac-Di-Sol |
| per tablet | 10 mEq or 840 mg | per tablet | 60 mg pregelatinized |
| | NaHCO₃ | | starch |
| | 25 mEq or 1590 mg | | 30 mg Klucel |
| | total buffer | | 15 mg mint flavor |
| | | | 15 mg magnesium |
| | | | stearate |

TABLE 9.G
Omeprazole (10 mg)-Clebopride (20 mg)
Bite-Disintegration Chewable Tablet

| | | Prokinetic | |
|---|---|---|---|
| PPI | Buffering Agent | Agent | Excipient |
| 10 mg | 15 mEq or 750 mg | 20 mg | 60 mg sucralose |
| omprazole | Ca(OH)₂ | Clebopride | 60 mg Ac-Di-Sol |
| per tablet | 10 mEq or 840 mg | per tablet | 60 mg pregelatinized |
| | NaHCO₃ | | starch |
| | 25 mEq or 1590 mg | | 30 mg Klucel |
| | total buffer | | 15 mg mint flavor |
| | | | 15 mg magnesium |
| | | | stearate |

TABLE 9.H
Omeprazole (40 mg)-Norcisapride (10 mg)
Bite-Disintegration Chewable Tablet

| | | Prokinetic | |
|---|---|---|---|
| PPI | Buffering Agent | Agent | Excipient |
| 40 mg | 15 mEq or 750 mg | 10 mg | 60 mg sucralose |
| micro- | Ca(OH)₂ | Norcisapride | 60 mg Ac-Di-Sol |
| encapsulated | 10 mEq or 840 mg | | 60 mg |
| omprazole | NaHCO₃ | | pregelatinized |
| per tablet | 25 mEq or 1590 mg | | starch |
| | total buffer | | 30 mg Klucel |
| | | | 15 mg mint flavor |
| | | | 15 mg magnesium |
| | | | stearate |

### Example 10

### Powder for Suspension Formulations

The following specific formuations are provided by way of reference only and are not intended to limit the scope of the invention. Each formulation contains therapeutically effective doses of PPI and prokinetic agent as well as sufficient buffering agent to prevent acid degredation of at least some of the PPI by raising the pH of gastric fluid.

TABLE 10.A
Omeprazole (20 mg) - Cisapride

| | 1 | 2 | 3 | 4 | 5 | 6 | 7 | 8 | 9 | 10 |
|---|---|---|---|---|---|---|---|---|---|---|
| Omeprazole | 20 | 20 | 20 | 20 | 20 | 20 | 20 | 20 | 20 | 20 |
| Cisapride | 2.5 | 5 | 10 | 20 | 20 | 10 | 5 | 2.5 | 10 | 15 |
| Sodium Bicarbonate | 1895 | 1680 | 1825 | 1895 | 1375 | 1650 | 1825 | 1650 | 1620 | 1600 |
| Xylitol 300 (sweetener) | 2000 | 2000 | 1500 | 1750 | 1750 | 2500 | 2000 | 1500 | 2000 | 2500 |
| Sucrose-powder (sweetener) | 1750 | 2000 | 2250 | 2000 | 2500 | 1500 | 1750 | 2500 | 2000 | 1500 |
| Sucralose (sweetener) | 125 | 100 | 150 | 75 | 100 | 70 | 80 | 130 | 125 | 80 |
| Xanthan Gum | 17 | 55 | 31 | 80 | 39 | 48 | 72 | 25 | 64 | 68 |
| Peach Flavor | 47 | 15 | 75 | 32 | 60 | 50 | 77 | 38 | 35 | 62 |
| Peppermint | 26 | 10 | 29 | 28 | 36 | 42 | 56 | 17 | 16 | 50 |
| Total Weight | 5880 | 5880 | 5880 | 5880 | 5880 | 5880 | 5880 | 5880 | 5880 | 5880 |

TABLE 10.B
Omeprazole (40 mg) - Metoclopramide

| | 1 | 2 | 3 | 4 | 5 | 6 | 7 | 8 | 9 | 10 |
|---|---|---|---|---|---|---|---|---|---|---|
| Omeprazole | 40 | 40 | 40 | 40 | 40 | 40 | 40 | 40 | 40 | 40 |
| Metoclopramide | 2.5 | 5 | 10 | 15 | 20 | 15 | 10 | 5 | 2.5 | 10 |
| Sodium Bicarbonate | 2010 | 1375 | 1680 | 1520 | 1400 | 1825 | 1680 | 1650 | 2030 | 1375 |
| Xylitol 300 (sweetener) | 1500 | 2750 | 2000 | 2500 | 2000 | 1750 | 2000 | 2500 | 1500 | 1750 |
| Sucrose-powder (sweetener) | 2000 | 1500 | 2000 | 1500 | 2250 | 2000 | 2000 | 1500 | 2000 | 2500 |
| Sucralose (sweetener) | 150 | 100 | 75 | 125 | 100 | 95 | 80 | 80 | 130 | 125 |
| Xanthan Gum 75 | 74 | 22 | 45 | 80 | 17 | 58 | 39 | 40 | 64 | 33 |
| Peach Flavor | 64 | 80 | 28 | 76 | 55 | 68 | 30 | 35 | 82 | 32 |
| Peppermint | 42 | 13 | 12 | 39 | 18 | 44 | 11 | 35 | 34 | 25 |
| Total Weight | 5880 | 5880 | 5880 | 5880 | 5880 | 5880 | 5880 | 5880 | 5880 | 5880 |

TABLE 10.C
Omeprazole (60 mg) - Ondasetron

| | 1 | 2 | 3 | 4 | 5 | 6 | 7 | 8 | 9 | 10 |
|---|---|---|---|---|---|---|---|---|---|---|
| Omeprazole | 60 | 60 | 60 | 60 | 60 | 60 | 60 | 60 | 60 | 60 |
| Ondasetron | 2.5 | 5 | 10 | 15 | 20 | 15 | 10 | 5 | 2.5 | 10 |
| Sodium Bicarbonate | 1750 | 2475 | 1310 | 2130 | 2005 | 1580 | 1110 | 2300 | 1325 | 1400 |
| Xylitol 300 (sweetener) | 2000 | 1500 | 2000 | 1500 | 2000 | 2500 | 2250 | 1500 | 1750 | 2500 |
| Sucrose-powder (sweetener) | 1750 | 1500 | 2250 | 2000 | 1500 | 1500 | 2250 | 1750 | 2500 | 1750 |
| Sucralose (sweetener) | 145 | 130 | 75 | 70 | 150 | 150 | 60 | 100 | 80 | 75 |
| Xanthan Gum 75 | 15 | 57 | 22 | 19 | 64 | 39 | 33 | 29 | 44 | 50 |
| Peach Flavor | 92 | 105 | 87 | 78 | 57 | 31 | 69 | 95 | 88 | 25 |
| Peppermint | 68 | 53 | 76 | 23 | 44 | 20 | 48 | 46 | 33 | 20 |
| Total Weight | 5880 | 5880 | 5880 | 5880 | 5880 | 5880 | 5880 | 5880 | 5880 | 5880 |

### Example 11

### Combination Tablet Delivering Bolus and Time-Released Doses of PPI

Tablets may be compounded using known methods by forming an inner core of 10 mg omeprazole powder, mixed with 750 mg sodium bicarbonate, and an outer core of 5-200 mg omeprazole enteric-coated granules and a therapeutically effective amount of a prokinetic agent mixed with known binders and excipients. Upon ingestion of the whole tablet, the tablet dissolves and the inner core is dispersed in the stomach where it is absorbed for immediate therapeutic effect. The enteric-coated granules are later absorbed in the duodenum to provide symptomatic relief later in the dosing cycle. This tablet is particularly useful in patients who experience breakthrough gastritis between conventional doses.

Modifications, equivalents, and variations of the present invention are possible in light of the teachings above, such that the invention may be embodied in other forms without departing from the spirit or essential characterstics of the invention. The present embodiments are therefore to be considered as illustrative and not restrictive, the scope of the invention being indicated by the appended claims rather than by the foregoing description. All changes that come within the meaning and range of equivalency of the claims are therefore intended to be embraced therein.

1. A pharmaceutical composition comprising:
(a) a therapeutically effective amount of at least one acid labile proton pump inhibitor;
(b) at least one buffering agent in an amount sufficient to increase gastric fluid pH to a pH that prevents acid degradation of at least some of the proton pump inhibitor in the gastric fluid; and
(c) a therapeutically effective amount of at least one prokinetic agent.

2. The composition of claim 1, wherein an initial serum concentration of the proton pump inhibitor is greater than about 100 ng/ml at any time within about 30 minutes after administration of the composition.

3. The composition of claim 1, wherein the proton pump inhibitor selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, leminoprazole; or a free base, free acid, salt, hydrate, ester, amide, enantiomer, isomer, tautomer, polymorph, or prodrug thereof.

4. The composition of claim 3, wherein the proton pump inhibitor is omeprazole or a free base, free acid, salt, hydrate, ester, amide, enantiomer, isomer, tautomer, polymorph, or prodrug thereof.

5. The composition of claim 1 comprising about 5 mg to about 200 mg of the proton pump inhibitor.

6. The composition of claim 1 comprising about 20 mg of the proton pump inhibitor.

7. The composition of claim 1 comprising about 40 mg of the proton pump inhibitor.

8. The composition of claim 1, wherein the composition is administered in an amount to maintain a serum concentration of the proton pump inhibitor greater than about 150 ng/ml from about 15 minutes to about 1 hour after administration of the composition.

9. The composition of claim 1, wherein upon oral administration to a subject, the composition provides a pharmacokinetic profile such that at least about 50% of total area under serum concentration time curve (AUC) for the proton pump inhibitor occurs within about 2 hours after administration of a single dose of the composition to the subject.

10. The composition of claim 1, wherein upon oral administration to the subject, the composition provides a pharmacokinetic profile such that the proton pump inhibitor reaches a maximum serum concentration within about 1 hour after administration of a single dose of the composition.

11. The composition of claim 1, wherein the proton pump inhibitor is microencapsulated with a material that enhances the shelf-life of the pharmaceutical composition.

12. The composition of claim 1, wherein the prokinetic agent is microencapsulated with a material that enhances the shelf-life of the pharmaceutical composition.

13. The composition of claim 11 or claim 12, wherein the material that enhances the shelf-life of the pharmaceutical composition is selected from the group consisting of cellulose hydroxypropyl ethers, low-substituted hydroxypropyl ethers, cellulose hydroxypropyl methyl ethers, ethylcellulose polymers, ethylcelluloses and mixtures thereof, polyvinyl alcohol, hydroxyethylcelluloses, carboxymethylcelluloses and salts of carboxymethylcelluloses, polyvinyl alcohol and polyethylene glycol co-polymers, monoglycerides, triglycerides, polyethylene glycols, modified food starch, acrylic polymers, mixtures of acrylic polymers with cellulose ethers, cellulose acetate phthalate, sepifilms, cyclodextrins, and mixtures thereof.

14. The composition of claim 12, wherein the material that enhances the shelf-life of the pharmaceutical composition is Klucel® or Nisso HPC.

15. The composition of claim 13, wherein the material that enhances the shelf-life of the pharmaceutical composition further comprises a buffering agent.

16. The composition of claim 1, wherein at least some of the prokinetic agent is coated.

17. The composition of claim 1, wherein some of the proton pump inhibitor is coated.

18. The composition of claim 16 or claim 17, wherein the coating is selected from a gastric resistant coating, a controlled-release coating, an enzymatic-controlled coating, a film coating, a sustained-release coating, an immediate-release coating, and a delayed-release coating.

19. The composition of claim 1, wherein the buffering agent is an alkaline earth metal salt or a Group IA metal selected from a bicarbonate salt of a Group IA metal, a carbonate salt of a Group IA metal.

20. The composition of claim 1, wherein the buffering agent is selected from sodium bicarbonate, calcium carbonate, magnesium hydroxide, and mixtures thereof.

21. The composition of claim 1, wherein the buffering agent is present in an amount of at least about 5 mEq.

22. The composition of claim 1, wherein the buffering agent is present in an amout from about 5 mEq to about 50 mEq.

23. The composition of claim 1 comprising from about 500 to about 3000 mg of buffering agent.

24. The composition of claim 1, wherein the prokinetic agent is selected from the group consisting of: 5-HT inhibitors; bulk forming agents; intraluminal agents; antimotility agents; saline laxatives; and luminally active osmotic agents.

25. The composition of claim 24, wherein the 5-HT inhibitor is a 5-HT₃ inhibitor or a 5-HT₄ inhibitor.

26. The composition of claim 24, wherein the prokinetic agent is selected from ondasetron, granisetron, dolanserton, cisapride, phylium, polycarbophil, fiber, bismuth, loperamide, clonidine, magnesium sulfate, sodium phosphate, mosapride, metoclopramide, domperidone, clebopride, erythromycin ethylsuccinate, erythromycin lactobionate, bethanechol, bethanechol chloride, norcisapride, and neostigmine; or a free base, free acid, salt, hydrate, ester, amide, enantiomer, isomer, tautomer, polymorph, or prodrug thereof.

27. The composition of claim 1, wherein the composition is in a dosage form selected from a powder, a tablet, a bite-disintegration tablet, a chewable tablet, a caplet, a capsule, an effervescent powder, a rapid-disintegration tablet, or an aqueous suspension produced from powder.

28. A method of treating a gastric acid related disorder in a subject by administering:
(a) a therapeutically effective amount of at least one acid labile proton pump inhibitor;
(b) at least one buffering agent in an amount sufficient to increase gastric fluid pH to a pH that prevents acid degradation of atl east some of the proton pump inhibitor in the gastric fluid; and
(c) a therapeutically effective amount of at least one prokinetic agent.

29. The method of claim 28, wherein the pharmaceutical composition is formulated for stomach delivery of at least some of the proton pump inhibitor.

30. The method of claim 28, wherein the gastric acid related disorder is duodenal ulcer disease, gastric ulcer disease, gastroesophageal reflux disease, erosive esophagitis, poorly responsive symptomatic gastroesophageal reflux disease, pathological gastrointestinal hypersecretory disease, Zollinger Ellison syndrome, heartburn, esophageal disorder, or acid dyspepsia.

31. The method of claim 28, wherein the proton pump inhibitor treats an episode of gastric acid related disorder.

32. The method of claim 28, wherein the proton pump inhibitor treats a medicament induced gastric acid related disorder.

33. The method of claim 28, wherein at least some of the proton pump inhibitor is microencapsulated.

34. The method of claim 28, wherein at least some of the prokinetic agent is microencapsulated or coated.

35. The method of claim 76, wherein the composition is in a dosage form selected from a powder, a tablet, a bite-disintegration tablet, a chewable tablet, a caplet, a capsule, an effervescent powder, a rapid-disintegration tablet, or an aqueous suspension produced from powder.
